(19) United States
(12) Patent Application Publication
(10) Pub. No.: US 2008/0312211 A1
(43) Pub. Date: Dec. 18, 2008

(54) Antibacterial Pyrrolopyridines, Pyrrolopyrimidines and Pyrroloazepines-154

(75) Inventors: Gregory Basarab, Waltham, MA (US); Haihong Ni, Waltham, MA (US); Brian Sherer, Waltham, MA (US); Fei Zhou, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/158,122

(22) PCT Filed: Dec. 19, 2006
(86) PCT No.: PCT/GB2006/004760

§ 371 Date: Jun. 19, 2008

Related U.S. Application Data
(60) Provisional application No. 60/753,527, filed on Dec. 23, 2005.

(51) Int. Cl. A61K 31/55; C07D 487/04; C07D 471/04; A61K 31/437; A61K 31/519; A61P 31/04

(52) U.S. Cl. 514/212.06; 544/280; 546/113; 540/521; 514/265.1; 514/300

(57) ABSTRACT

Compounds of formula (I) and their pharmaceutically acceptable salts are described: formula (I). Processes for their preparation, pharmaceutical compositions containing them, their use as medicaments and their use in the treatment of bacterial infections are also described.

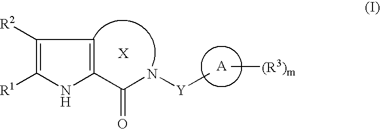

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm-blooded animals such as humans.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D.C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomerase IV are known in the art. For example, coumarin-containing compounds are described in patent application number WO 99/35155, 5,6-bicyclic heteroaromatic compounds are described in patent application WO 02/060879, pyrazole compounds are described in patent application WO 01/52845 (U.S. Pat. No. 6,608,087), and pyrrole compounds described in patent application WO 05/026149.

We have discovered a new class of compounds which are useful for inhibiting DNA gyrase and topoisomerase IV.

Therefore the present invention provides a compound of formula (I):

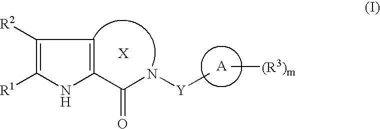

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)a wherein a is 0 to 2 and C₃₋₆cycloalkyl; wherein R² may be optionally substituted on carbon by one or more halo or C₃₋₆cycloalkyl;

R³ represents a substituent on carbon and is selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂-amino, C₁₋₄alkanoylamino, N—(C₁₋₄alkyl)carbamoyl, N,N—(C₁₋₄alkyl)₂-carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)a wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R⁴— or heterocyclyl-R⁵—; wherein R³ may be optionally substituted on carbon by one or more R⁶; and wherein if said heterocyclyl contains an —NH— moiety that nitrogen may be optionally substituted by a group selected from R⁷;

R⁶ is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)a wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹³— or heterocyclyl-R¹⁴—; wherein R⁶ may be optionally substituted on carbon by one or more R¹⁵; and wherein if said heterocyclyl contains an —NH— moiety that nitrogen may be optionally substituted by a group selected from R¹⁶;

R⁴, R⁵, R¹³ and R¹⁴ are independently selected from a direct bond, —O—, —N(R⁸)—, —C(O)—, —N(R⁹)C(O)—, —C(O)N(R¹⁰)—, —S(O)ₚ—, —SO₂N(R¹¹)— or —N(R¹²)SO₂—; wherein R⁸, R⁹, R¹⁰, R¹¹ and R¹² are independently selected from hydrogen or C₁₋₄alkyl and p is 0-2;

R¹⁵ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

Ring X is a heterocyclic ring selected from X¹, X², X³ and X⁴;

X¹ is

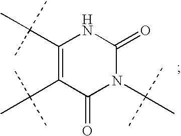

X² is

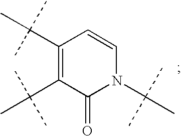

X³ is

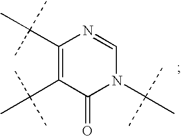

X⁴ is

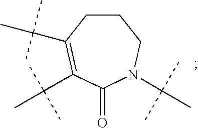

Y is selected from phenyl, azetidinyl, piperidinyl and pyrrolidinyl; wherein the N of said azetidinyl, piperidinyl and pyrrolidinyl ring is directly attached to Ring A; and further wherein Y may be optionally substituted on carbon by one or two halo, C₁₋₄alkyl or C₁₋₄alkoxy;

Ring A is carbocyclyl or heterocyclyl; wherein if said heterocyclyl contains an —NH— moiety that nitrogen may be optionally substituted by a group selected from R¹⁷;

m is 0-4; wherein the values of R³ may be the same or different;

R⁷, R¹⁶ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; or a pharmaceutically acceptable salt thereof.

In this specification the term alkyl includes both straight and branched chain alkyl groups. For example, “C₁₋₄alkyl” includes methyl, ethyl, propyl, isopropyl and t-butyl. However references to individual alkyl groups such as propyl are specific for the straight chain version only. An analogous convention applies to other generic terms.

Where optional substituents are chosen from one or more groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring nitrogen and/or a ring sulphur atom may be optionally oxidised to form the N- or S-oxide(s). In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. Further examples and suitable values of the term “heterocyclyl” are thiazolyl, quinolinyl, benzothiazolyl, pyrimidinyl and pyridinyl.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxycarbonylamino” include methoxycarbonylamino, ethoxycarbonylamino, n- and t-butoxycarbonylamino. Examples of “C₁₋₄alkoxy” include methoxy, ethoxy and propoxy. Examples of “C₁₋₄alkanoylamino” include formamido, acetamido and propionylamino. Examples of “C₁₋₄alkylS(O)a wherein a is 0 to 2” include methylthio, ethylthio, methylsulphinyl, ethylsulphinyl, mesyl and ethylsulphonyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂amino” include di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)sulphamoyl” are N-(methyl)sulphamoyl and N-(ethyl)sulphamoyl. Examples of “N,N—(C₁₋₄alkyl)₂sulphamoyl” are N,N-(dimethyl)sulphamoyl and N-(methyl)-N-(ethyl)sulphamoyl. Examples of “N—(C₁₋₄alkyl)carbamoyl” are methylaminocarbonyl and ethylaminocarbonyl. Examples of “N,N—(C₁₋₄alkyl)₂carbamoyl” are dimethylaminocarbonyl and methylethylaminocarbonyl. Examples of “N—(C₁₋₄alkoxy)carbamoyl” are methoxyaminocarbonyl and isopropoxyaminocarbonyl. Examples of “N—(C₁₋₄alkyl)-N—(C₁₋₄alkoxy)carbamoyl” are N-methyl-N-methoxyaminocarbonyl and N-methyl-N-ethoxyaminocarbonyl. Examples of “C₃₋₆cycloalkyl” are cyclopropyl, cyclobutyl, cyclopropyl and cyclohexyl. Examples of “N′—(C₁₋₄alkyl)ureido” are N′-methylureido and N′-isopropylureido. Examples of “N′N′—(C₁₋₄alkyl)₂ureido” are N′N′-dimethylureido and N′-methyl-N′-isopropylureido. Examples of “N—(C₁₋₄alkyl)hydrazinocarbonyl” are N′-methylhydrazinocarbonyl and N′-isopropylhydrazinocarbonyl. Examples of “N′,N′—(C₁₋₄alkyl)₂hydrazinocarbonyl” are N′N′-dimethylhydrazinocarbonyl and N′-methyl-N′-isopropylhydrazinocarbonyl. Examples of “C₁₋₄alkylsulphonylamino” include methylsulphonylamino, isopropylsulphonylamino and t-butylsulphonylamino. Examples of “C₁₋₄alkylsulphonylaminocarbonyl” include methylsulphonylaminocarbonyl, isopropylsulphonylaminocarbonyl and t-butylsulphonylaminocarbonyl. Examples of “C₁₋₄alkylsulphonyl” include methylsulphonyl, isopropylsulphonyl and t-butylsulphonyl.

The hatched lines in the structures for X¹, X², X³ and X⁴ show the points of attachment of each of X¹, X², X³ and X⁴ to the rest of the molecule. For example, when ring X is X¹, then the structure of formula (I) is as follows:

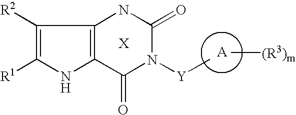

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that certain compounds of formula (I) contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase and/or topoisomerase IV, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase and/or topoisomerase IV by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

R¹ is C₁₋₄alkyl.

R¹ is methyl.

R² is halo or cyano.

R² is chloro or cyano.

R² is halo.

R² is chloro.

R² is cyano.

Ring X is a heterocyclic ring selected from X¹, X² or X⁴.

Ring X is X¹.

Ring X is X².

Ring X is X³.

Ring X is X⁴.

Y is phenyl; wherein Y may be optionally substituted on carbon by one or two halo, C₁₋₄alkyl or C₁₋₄alkoxy.

Y is azetidinyl; wherein the N of said azetidinyl ring is directly attached to Ring A; and further wherein Y may be optionally substituted on carbon by one or two halo, C₁₋₄alkyl or C₁₋₄alkoxy.

Y is piperidinyl; wherein the N of said piperidinyl ring is directly attached to Ring A; and further wherein Y may be optionally substituted on carbon by one or two halo, C₁₋₄alkyl or C₁₋₄alkoxy.

Y is pyrrolidinyl; wherein the N of said pyrrolidinyl ring is directly attached to Ring A; and further wherein Y may be optionally substituted on carbon by one or two halo, C₁₋₄alkyl or C₁₋₄alkoxy.

Y is selected from phenyl and piperidinyl; wherein the N of said piperidinyl ring is directly attached to Ring A; and further wherein Y may be optionally substituted on carbon by one halo or C₁₋₄alkoxy.

Y is selected from phenyl and piperidinyl; wherein the N of said piperidinyl ring is directly attached to Ring A; and further wherein Y may be optionally substituted on carbon by one fluoro or methoxy.

Y is selected from phenyl, 3-fluoropiperidinyl and 3-methoxypiperidinyl; wherein the N of said piperidinyl ring is directly attached to Ring A.

Y is selected from phenyl, (3S,4R)-3-fluoropiperidinyl and 3-methoxypiperidinyl; wherein the N of said piperidinyl ring is directly attached to Ring A.

Ring A is carbocyclyl or heterocyclyl.

Ring A is phenyl, thiazolyl, benzothiazolyl, pyrimidinyl or pyridinyl.

Ring A is phenyl, thiazol-2-yl, benzothiazol-2-yl, pyrimidin-4-yl or pyridin-2-yl.

Ring A is carbocyclyl.

Ring A is phenyl.

Ring A is heterocyclyl; wherein if said heterocyclyl contains an —NH— moiety that nitrogen may be optionally substituted by a group selected from R¹¹.

Ring A is heterocyclyl.

Ring A is thiazolyl, quinolinyl, benzothiazolyl, pyrimidinyl or pyridinyl.

Ring A is thiazol-2-yl, quinolin-4-yl, benzothiazol-2-yl, pyrimidin-4-yl, pyridin-2-yl or pyridin-4-yl.

R³ represents a substituent on carbon and is selected from halo, carboxy, C₁₋₄alkyl, C₁₋₄alkoxy or C₁₋₄alkoxycarbonyl; wherein R³ may be optionally substituted on carbon by one or more R⁶; wherein R⁶ is selected from C₁₋₄alkoxy.

R³ represents a substituent on carbon and is selected from fluoro, chloro, carboxy, methyl, methoxy, methoxycarbonyl, ethoxycarbonyl or isopropoxycarbonyl; wherein R³ may be optionally substituted on carbon by one or more R⁶; wherein R⁶ is selected from methoxy.

R³ represents a substituent on carbon and is selected from fluoro, chloro, carboxy, methyl, methoxy, methoxycarbonyl, methoxymethyl, ethoxycarbonyl or isopropoxycarbonyl.

R³ is selected from halo, carboxy, carbamoyl, C₁₋₄alkoxy or C₁₋₄alkoxycarbonyl, N—(C₁₋₄alkyl)carbamoyl, N,N—(C₁₋₄alkyl)₂carbamoyl, N—(C₁₋₄alkoxy)carbamoyl, or N—(C₁₋₄alkyl)-N—(C₁₋₄alkoxy)carbamoyl.

R³ is selected from carboxy, carbamoyl, C₁₋₄alkoxy or C₁₋₄alkoxycarbonyl, N—(C₁₋₄alkyl)carbamoyl, N,N—(C₁₋₄alkyl)₂carbamoyl, N—(C₁₋₄alkoxy)carbamoyl, or N—(C₁₋₄alkyl)-N—(C₁₋₄alkoxy)carbamoyl. R³ is selected from halo, carboxy, carbamoyl, C₁₋₄alkoxy or C₁₋₄alkoxycarbonyl.

R³ is selected from carboxy, carbamoyl, C₁₋₄alkoxy or C₁₋₄alkoxycarbonyl.

R³ is selected from halo, carboxy, C₁₋₄alkoxy or C₁₋₄alkoxycarbonyl.

R³ is selected from carboxy, C₁₋₄alkoxy or C₁₋₄alkoxycarbonyl.

R³ is selected from carboxy, carbamoyl and C₁₋₄alkoxycarbonyl and m is 1 or 2.

R³ is selected from carboxy and C₁₋₄alkoxycarbonyl and m is 1 or 2.

m is 1 or 2.

m is 1.

m is 2.

R³ is a substituent on carbon and is selected from halo, carboxy, carbamoyl, C₁₋₄alkyl, C₁₋₄alkoxy, N—(C₁₋₄alkyl)carbamoyl, N—(C₁₋₄alkoxy)carbamoyl or C₁₋₄alkoxycarbonyl; wherein R³ may be optionally substituted on carbon by one or more R⁶; wherein

R⁶ is selected from C₁₋₄alkoxy or carbocyclyl-R⁴—; and

R⁴ is a direct bond.

R³ is a substituent on carbon and is selected from chloro, carboxy, carbamoyl, methyl, methoxy, N-(isopropyl)carbamoyl, N-(methoxy)carbamoyl, methoxycarbonyl or ethoxycarbonyl; wherein R³ may be optionally substituted on carbon by one or more R⁶; wherein

R⁶ is selected from methoxy or phenyl-R⁴—; and

R⁴ is a direct bond.

R³ is a substituent on carbon and is selected from chloro, carboxy, carbamoyl, methyl, methoxymethyl, methoxy, N-(1-methyl-1-phenylethyl)carbamoyl, N-(methoxy)carbamoyl, methoxycarbonyl or ethoxycarbonyl.

R⁶ is hydrogen.

m is 1 or 2; wherein the values of R³ may be the same or different.

Therefore in a further aspect of the invention there is provided a compound of formula (I) (as depicted above) wherein:

R¹ is C₁₋₄alkyl;

R² is halo or cyano;

Ring X is a heterocyclic ring selected from X¹, X² or X⁴;

Y is selected from phenyl and piperidinyl; wherein the N of said piperidinyl ring is directly attached to Ring A; and further wherein Y may be optionally substituted on carbon by one halo or C₁₋₄alkoxy;

Ring A is carbocyclyl or heterocyclyl;

R³ represents a substituent on carbon and is selected from halo, carboxy, C₁₋₄alkyl, C₁₋₄alkoxy or C₁₋₄alkoxycarbonyl; wherein R³ may be optionally substituted on carbon by one or more R⁶; wherein R⁶ is selected from C₁₋₄alkoxy; and

m is 1 or 2; wherein the values of R³ may be the same or different;

or a pharmaceutically acceptable salt thereof.

Therefore in a further aspect of the invention there is provided a compound of formula (I) (as depicted above) wherein:

R¹ is methyl;

R² is chloro or cyano;

Ring X is a heterocyclic ring selected from X¹, X² or X⁴;

Y is selected from phenyl, 3-fluoropiperidinyl and 3-methoxypiperidinyl; wherein the N of said piperidinyl ring is directly attached to Ring A;

Ring A is phenyl, thiazol-2-yl, benzothiazol-2-yl, pyrimidin-4-yl or pyridin-2-yl;

R³ represents a substituent on carbon and is selected from fluoro, chloro, carboxy, methyl, methoxy, methoxycarbonyl, methoxymethyl, ethoxycarbonyl or isopropoxycarbonyl; and

m is 1 or 2; wherein the values of R³ may be the same or different;

or a pharmaceutically acceptable salt thereof.

Particular compounds of the invention are the compounds of the Examples, each of which provides a further independent aspect of the invention. In further aspects, the present invention also comprises any two or more compounds of the Examples.

In one embodiment of the invention are provided compounds of formula (I), in an alternative embodiment are provided pharmaceutically-acceptable salts of compounds of formula (I).

In a further aspect the present invention provides a process for preparing a compound of formula (I) or a pharmaceutically-acceptable salt thereof.

Thus, the present invention also provides that the compounds of the formula (I) and pharmaceutically-acceptable salts thereof can be prepared by a process as follows (wherein the variables are as defined above unless otherwise stated):

Process a) for compounds of formula (I) wherein X═X¹, X², X³, and X⁴; cyclizing a compound of formula (II):

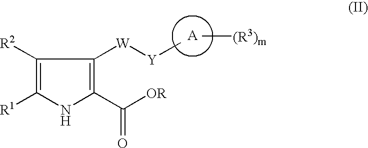

wherein R═C₁₋₄alkyl or hydrogen; W═—NC(O)N, —CH═CHNH—, —N═CH—NH— or —(CH₂)₃—NH— (respectively) into a compound of formula (I); or
Process b) for compounds of formula (I) wherein X═X²; converting a compound of formula (III):

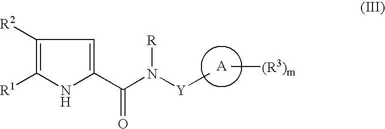

wherein R≡—CH₂C(OCH₂CH₂O) into a compound of formula (I); or
Process c) for compounds of formula (I) wherein Y=phenyl; reacting a compound of formula (IV):

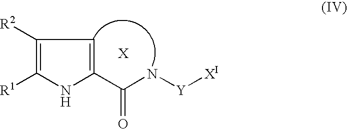

with a compound of formula (V):

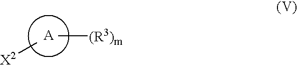

wherein one of X¹ and X² is a displaceable group “L” and the other is an organometallic reagent “M”; or
Process d) for compounds of formula (I) wherein Y=azetidinyl, piperidinyl or pyrrolidinyl linked to Ring A via the nitrogen in the ring; reacting a compound of formula (VI):

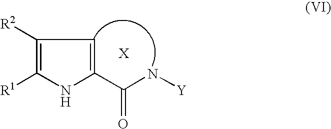

with a compound of formula (VII):

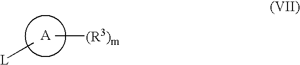

wherein D is a displaceable group;
and thereafter if necessary:
i) converting a compound of the formula (I) into another compound of the formula (I);
ii) removing any protecting groups;
iii) forming a pharmaceutically acceptable salt.

L is a displaceable group, suitable values for L include chloro, bromo, tosyl and trifluoromethylsulphonyloxy.

M is an organometallic reagent, suitable values for M include organoboron and organotin reagents, in particular B(OR^{z})₂ where R^{z} is hydrogen or C₁₋₆alkyl for example B(OH)₂; and Sn(R^{y})₃ where R^{y} is C₁₋₆alkyl for example Sn(Bu)₃.

D is a displaceable group, suitable values for L include halo, including chloro and bromo.

Specific reaction conditions for the above reactions are as follows.

Process a) Compounds of formula (II) may be cyclized in a suitable solvent, for example toluene, N-methylpyrrolidine or xylene with heat and preferably pressure.

Compounds of formula (II) may be prepared by the following scheme:

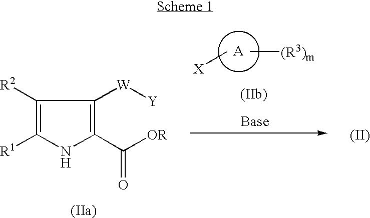

Compounds of formula (IIa) and (IIb) are commercially available compounds, or they are known in the literature or they may be prepared by standard processes known in the art.

Process b) Compounds of formula (III) may be cyclized in the presence of acid such as methanesulfonic acid and heat with a suitable solvent such as toluene where R═CH₂(—OCH₂CH₂O—)

Compounds of formula (III) may be prepared by the following scheme:

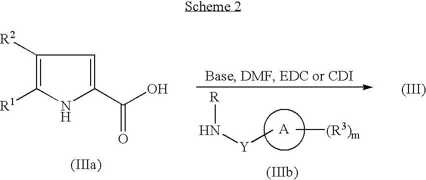

Compounds of formula (IIIa) and (IIIb) are commercially available compounds, or they are known in the literature or they may be prepared by standard processes known in the art.

Process c) Compounds of formula (IV) and (V) may be reacted together by coupling chemistry utilizing an appropriate catalyst. Such reactions are well known in the art. For example, where M is an organoboron group, Pd(PPh₃)₄ and a suitable base such as sodium carbonate can be utilized. In the case where M is an organotin reagent, Pd(PPh₃)₄ can be utilized as the catalyst. The reactions take place in suitable solvents and may require thermal conditions.

Compounds of formula (IV) may be prepared by the following scheme:

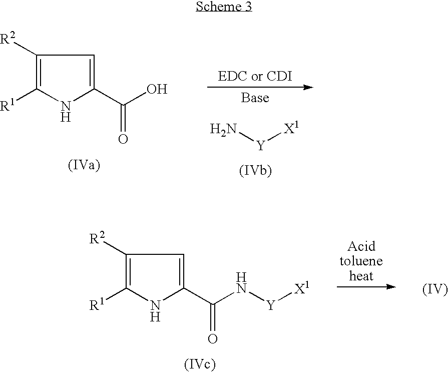

Compounds of formula (IVa), (IVb) and (V) are commercially available compounds, or they are known in the literature or they may be prepared by standard processes known in the art.

Process d) Compounds of formula (VI) and (VII) may be reacted together in a suitable solvent, for example N-methylpyrrolidine or DMF with heat and a suitable base, for example triethylamine or Hunig's base.

Compounds of formula (VI) may be prepared by the following scheme:

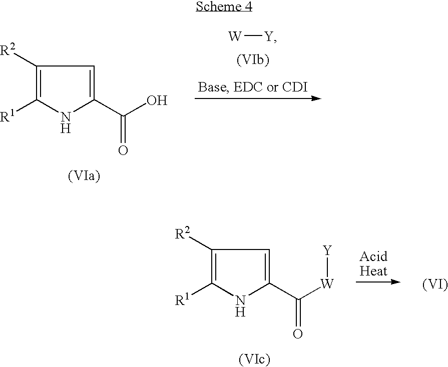

Compounds of formula (VIa), (VIb) and (VII), are commercially available compounds, or they are known in the literature or they may be prepared by standard processes known in the art.

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

Compounds were tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays were performed in multiwell plates in 100 μl reactions containing: 50 mM TRIS buffer pH 7.5, 75 mM ammonium acetate, 5.5 mM magnesium chloride, 0.5 mM ethylenediaminetetraacetic acid, 5% glycerol, 1 mM 1,4-Dithio-DL-threitol, 200 nM bovine serum albumin, 16 μg/ml sheared salmon sperm DNA, 4 nM E. coli GyrA, 4 nM E. coli GyrB, 250 μM ATP, and compound in dimethylsulfoxide. Reactions were quenched with 150 μl of ammonium molybdate/malachite green detection reagent containing 1.2 mM malachite green hydrochloride, 8.5 mM ammonium molybdate tetrahydrate, and 1 M hydrochloric acid. Plates were read in an absorbance plate reader at 625 nm and percent inhibition values were calculated using dimethylsulfoxide (2%)-containing reactions as 0% inhibition and novobiocin-containing (2 μM) reactions as 100% inhibition controls.

Compounds were tested for inhibition of topoisomerase IV ATPase activity as described above for GyrB except the 100 μl reactions contained the following: 20 mM TRIS buffer pH 8, 50 mM ammonium acetate, 8.5 mM magnesium chloride, 0.5 mM ethylenediaminetetraacetic acid, 5% glycerol, 5 mM 1,4-Dithio-DL-threitol, 0.005% Brij-35, 5 μg/ml sheared salmon sperm DNA, 10 nM E. Coli ParC, 10 nM E. coli ParE, 150 μM ATP, and compound in dimethylsulfoxide. Compound potency was based on IC₅₀ measurements determined from reactions performed in the presence of 10 different compound concentrations.

Compounds of the invention generally have IC₅₀ values of <200 μg/ml in one or both assays described hereinabove.

### Bacterial Susceptibility Testing Methods

Compounds were tested for antimicrobial activity by susceptibility testing in liquid media. Compounds were dissolved in dimethylsulfoxide and tested in 10 doubling dilutions in the susceptibility assays. The organisms used in the assay were grown overnight on suitable agar media and then suspended in a liquid medium appropriate for the growth of the organism. The suspension was a 0.5 McFarland and a further 1 in 10 dilution was made into the same liquid medium to prepare the final organism suspension in 100 μL. Plates were incubated under appropriate conditions at 37 degrees C. for 24 hrs prior to reading. The Minimum Inhibitory Concentration was determined as the lowest drug concentration able to reduce growth by 80% or more.

Example 22 had an MIC of 50 nM/ml against Streptococcus pneumoniae.

According to a further feature of the invention there is provided a compound of the formula (I), or a pharmaceutically-acceptable salt thereof for use in a method of treatment of the human or animal body by therapy.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention the compounds of the invention inhibit topoisomerase IV and are therefore of interest for their antibacterial effects. In one aspect of the invention the compounds of the invention inhibit both DNA gyrase and topoisomerase IV and are therefore of interest for their antibacterial effects.

It is expected that the compounds of the present invention will be useful in treating bacterial infections including, but not limited to community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I) or a pharmaceutically acceptable salt thereof as defined hereinbefore.

A further feature of the present invention is a compound of formula (I) and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for use in the production of an anti-bacterial effect in a warm-blooded animal such as a human being.

According to a further aspect of the invention there is provided the use of a compound of formula (I), or a pharmaceutically acceptable salt thereof in the manufacture of a medicament for use in inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof in the manufacture of a medicament for use in the treatment of a bacterial infection in a warm-blooded animal such as a human being.

According to a further aspect of the invention there is provided a compound of formula (I), or a pharmaceutically acceptable salt thereof for use in the production of an anti-bacterial effect in a warm-blooded animal such as a human being.

According to a further aspect of the invention there is provided a compound of formula (I), or a pharmaceutically acceptable salt thereof for use in inhibition of bacterial DNA gyrase and/or topoisomerase IV in a warm-blooded animal such as a human being.

Thus according to a further aspect of the invention there is provided a compound of formula (I), or a pharmaceutically acceptable salt thereof for use in the treatment of a bacterial infection in a warm-blooded animal such as a human being.

In order to use a compound of the formula (I) or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I) or a pharmaceutically-acceptable salt thereof, and a pharmaceutically-acceptable diluent or carrier.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in inhibition of bacterial DNA gyrase and/or topoisomerase IV in an warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a bacterial infection in an warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention the pharmaceutical composition of this invention may also contain or be co-administered (simultaneously, sequentially or separately) with one or more known drugs selected from other clinically useful antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

In addition to its use in therapeutic medicine, compounds of formula (I) and their pharmaceutically acceptable salts are also useful as pharmacological tools in the development and standardisation of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above, other pharmaceutical composition, process, method, use and medicament manufacture features, the alternative and particular embodiments of the compounds of the invention described herein also apply.

### EXAMPLES

The invention is now illustrated but not limited by the following Examples in which unless otherwise stated:—

(i) evaporations were carried out by rotary evaporation in-vacuo and work-up procedures were carried out after removal of residual solids by filtration;
(ii) reactions were carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;
(v) the structure of the end-products of the invention were generally confirmed by NMR and mass spectral techniques [proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected]or using Agilent 1100series LC/MSD equipped with Sedex 75ELSD, run in APCI mode and, where appropriate, either positive ion data or negative ion data were collected; optical rotations were determined at 589 nm at 20° C. using a Perkin Elmer Polarimeter 341;
(vi) each intermediate was purified to the standard required for the subsequent stage and was characterised in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) the following abbreviations have been used:
DMF N,N-dimethylformamide;
TLC thin layer chromatography;
HPLC high pressure liquid chromatography;
DMSO dimethylsulfoxide;
CDCl₃ deuterated chloroform;
MS mass spectroscopy;
APCI atmospheric pressure chemical ionisation;
EtOAc ethyl acetate;
MeOH methanol;
TFA trifluoroacetic acid;
HATU N-[(dimethylamino)-1H,2,3-triazolo[4,5-b-]pyridin-1-ylmethylene]-N-methylmethanaminium hexafluorophosphate N-oxide;
THF tetrahydrofuran;
EtOH ethanol;
DCM or CH₂Cl₂ dichloromethane;
Et₃N triethylamine;
DME dimethoxyethane;
   (viii) temperatures are quoted as ° C.; and
   (ix) GCMS is Gas phase chromatography (model 6890N) with Mass Spectrometer(model 5973) manufactured by Agilent and was used according to manufacturers instructions.

If there is no synthetic procedure given for a particular material, the material is either commercially available or can be synthesized from known procedures.

### Example 1

### Methyl 2-[4-(7-cyano-6-methyl-2,4-dioxo-1,2,4,5-tetrahydro-3H-pyrrolo[3,2-d]pyrimidin-3-yl)piperidin-1-yl]-1,3-thiazole-5-carboxylate

6-Methyl-2,4-dioxo-3-piperidin-4-yl-2,3,4,5-tetrahydro-1H-pyrrolo[3,2-d]pyrimidine-7-carbonitrile (Intermediate 1) (35 mg, 0.128 mmol) was suspended in dry DMF (4 ml). Triethylamine (13 mg, 0.128 mmol) and methyl 2-bromo-1,3-thiazole-5-carboxylate (commercially available) (29 mg, 0.128 mmol) were added, the mixture was then heated under microwave at 130° C. for 30 minutes. The mixture was diluted with ethyl acetate (50 ml), washed with water (2×20 ml), the organic layer was dried over magnesium sulfate, concentrated and purified by column chromatography (5% methanol in dichloromethane) giving the desired product as an off-white solid (17 mg). MP>345° C. (dec.).

MS (ES): 415.08 (MH⁺) for C₁₈H₁₈N₆O₄S

¹H-NMR δ: 1.67 (m, 2H); 2.36 (s, 3H); 2.64 (m, 2H); 3.32 (m, 2H); 3.75 (s, 3H); 4.07 (m, 2H); 5.04 (m, 1H); 7.87 (s, 1H); 11.81 (br, 1H); 12.80 (br, 1H).

### Example 2

### Ethyl 2-[4-(7-cyano-6-methyl-2,4-dioxo-1,2,4,5-tetrahydro-3H-pyrrolo[3,2-d]pyrimidin-3-yl)piperidin-1-yl]-1,3-benzothiazole-7-carboxylate

Example 2 was synthesized following the procedure in Example 1 from Intermediate 1 and ethyl 2-bromo-1,3-benzothiazole-7-carboxylate (prepared as described in U.S. Pat. No. 5,770,758).

MS (ES): 479.01 (MH⁺) for C₂₃H₂₂N₆O₄S

¹H-NMR (CDCl₃+5 drop of CD₃OD) δ: 1.40 (t, 3H); 1.74 (m, 2H); 2.40 (s, 3H); 2.73 (m, 2H); 3.23 (t, 2H); 4.30 (m, 2H); 4.41 (q, 2H); 5.09 (m, 1H); 7.33 (t, 1H); 7.64 (d, 1H); 7.75 (d, 1H).

### Example 3

### 2-[4-(7-Cyano-6-methyl-2,4-dioxo-1,2,4,5-tetrahydro-3H-pyrrolo[3,2-d]pyrimidin-3-yl)piperidin-1-yl]-1,3-thiazole-5-carboxylic acid

Methyl 2-[4-(7-cyano-6-methyl-2,4-dioxo-1,2,4,5-tetrahydro-3H-pyrrolo[3,2-d]pyrimidin-3-yl)piperidin-1-yl]-1,3-thiazole-5-carboxylate, prepared in Example 1 (12 mg, 0.029 mmol), was dissolved in a mixture of THF/methanol/H₂O (2:1:1, 4 ml). NaOH (0.5 ml, 2M) was added and the mixture was stirred at room temperature overnight. Solvent was removed under vacuum and the remaining aqueous solution was acidified to pH=2, the white precipitate which formed was filtered, washed with water and collected as the desired product (3 mg).

MS (ES): 401 (MH⁺) for C₁₇H₁₆N₆O₄S

¹H-NMR δ: 1.67 (m, 2H); 2.36 (s, 3H); 2.64 (m, 2H); 3.32 (m, 2H); 4.10 (m, 2H); 5.04 (m, 1H); 7.77 (s, 1H); 11.81 (br, 1H); 12.81 (br, 1H); 13.30 (br, 1H).

### Examples 4-5

The following Examples 4-5 were prepared by a procedure analogous to that of Example 3 using the starting materials indicated.

| Ex | Compound | Data | SM |
|---|---|---|---|
| 4 | 2-[4-(7-Cyano-6-methyl-2,4- | MS (ES): 450.99 (MH⁺) for | Example 2 |
| | dioxo-1,2,4,5-tetrahydro-3H- | C₂₁H₁₈N₆O₄S; NMR δ: 1.70 (m, 2H); | |
| | pyrrolo[3,2-d]pyrimidin-3- | 2.40 (s, 3H); 2.70 (m, 2H); 3.23 (m, | |
| | yl)piperidin-1-yl]-1,3- | 2H); 4.23 (m, 2H); 5.05 (m, 1H); | |
| | benzothiazole-7-carboxylic acid | 7.42 (t, 1H); 7.68 (d, 1H); 7.70 (d, 1H); | |
| | | 11.79 (s, 1H); 12.80 (s, 1H) | |
| 5 | 6-[4-(7-Cyano-6-methyl-2,4- | MS (ES): 426.06 (MH⁺) for | Example |
| | dioxo-1,2,4,5-tetrahydro-3H- | C₁₉H₁₉N₇O₅; NMR_δ: 1.62 (m, 2H); | 28 |
| | pyrrolo[3,2-d]pyrimidin-3- | 2.36 (s, 3H); 2.50 (m, 2H); 3.17 (m, | |
| | yl)piperidin-1-yl]-2- | 2H); 3.50 (m, 2H); 3.86 (s, 3H); | |
| | methoxypyrimidine-4- | 5.05 (m, 1H); 7.04 (s, 1H); 11.79 (s, 1H); | |
| | carboxylic acid | 12.80 (s, 1H) | |

### Example 6

### 4′-(7-Cyano-6-methyl-2,4-dioxo-1,2,4,5-tetrahydro-3H-pyrrolo[3,2-a]pyrimidin-3-yl)biphenyl-3-carboxylic acid

3-(4-Bromophenyl)-6-methyl-2,4-dioxo-2,3,4,5-tetrahydro-1H-pyrrolo[3,2-d]pyrimidine-7-carbonitrile (Intermediate 4) (50 mg, 0.15 mmol), (3-ethoxycarbonylphenyl)boronic acid (30 mg, 0.15 mmol), PS—PPh₃-Pd (resin, 136 mg, 0.11 mmol/g, 0.015 mmol) and potassium carbonate (69 mg, 0.5 mmol) were suspended in DME/EtOH/H₂O (2:2:1, 4 ml) in a micro-reaction tube. The mixture was heated to 120° C. in a microwave for 30 minutes and then cooled to room temperature. The reaction mixture was filtered and the filter cake was washed with water (5 ml). The combined aqueous filtrates were washed with diethyl ether. The aqueous layer was acidified (2M HCl) to pH=2 and extracted with ethyl acetate (2×10 ml). The combined organic layers were concentrated and purified by flash column chromatography eluted with 15% methanol in dichloromethane to give the desired product as an off-white solid (32 mg).

MS (ES): 387 (MH⁺) for C₂₁H₁₄N₄O₄

¹H-NMR δ: 2.40 (s, 3H); 7.38 (d, 2H); 7.67 (t, 1H); 7.80 (d, 2H); 7.96 (d, 2H); 8.24 (s, 1H); 12.05 (s, 1H); 12.96 (s, 1H); 13.12 (br, 1H).

### Example 7

The following Example 7 was prepared by a procedure analogous to that of Example 6 using the starting materials indicated.

| Ex | Compound | Data | SM |
|---|---|---|---|
| 7 | 4′-(7-Cyano-6-methyl-2,4- | MS (ES): 405 (MH⁺) for | Intermediate 4 and |
| | dioxo-1,2,4,5-tetrahydro- | C₂₁H₁₃N₄O₄F | 5-(dihydroxy- |
| | 3H-pyrrolo[3,2- | NMR δ: 2.37 (s, 3H); 7.35 (d, 2H); | boryl)-2- |
| | d]pyrimidin-3-yl)-4- | 7.42 (t, 1H); 7.73 (d, 2H); 7.98 (m, | fluorobenzoic acid |
| | fluorobiphenyl-3- | 1H); 8.10 (d, 1H); 12.00 (s, 1H); | |
| | carboxylic acid | 12.92 (s, 1H); 13.38 (br, 1H) | |

### Example 8

### Ethyl 2-[4-(3-chloro-2-methyl-7-oxo-1,7-dihydro-6H-pyrrolo[2,3-c]pyridin-6-yl)piperidin-1-yl]isonicotinate

3-Chloro-2-methyl-6-piperidin-4-yl-1,6-dihydro-7H-pyrrolo[2,3-c]pyridin-7-one (Intermediate 6) (40 mg, 0.133 mol), ethyl-2-fluoroisonicotinate (22.5 mg, 0.133 mmol) and diisopropylethylamine (34.4 mg, 0.266 mmol) were mixed in N-methylpyrrolidone (3 ml) in a microwave reaction tube and heated to 160° C. When the reaction was complete, the mixture was diluted with ethyl acetate (10 ml) and washed with water (3×5 ml), the organic layer was concentrated and purified by column chromatography, eluting with 5% methanol in dichloromethane and to give the desired product as a solid (15 mg).

MS (ES): 415 (MH⁺) for C₂₁H₂₃ClN₄O₃

¹H-NMR (CDCl₃) δ: 1.33 (t, 3H); 2.0 (m, 4H); 2.42 (s, 3H); 3.06 (m, 2H); 4.30 (q, 2H); 4.60 (m, 2H); 5.30 (m, 1H); 6.52 (d, 1H); 6.92 (d, 1H); 7.10 (d, 1H); 7.30 (s, 1H); 8.30 (d, 1H); 12.02 (s, 1H).

### Examples 9-15

Examples 9-15 were prepared by a procedure analogous to that of Example 8 using the starting materials indicated.

| Ex | Compound | Data | SM |
|---|---|---|---|
| 9 | Methyl 2-[(3,4)-cis-4- | MS (ES): 437 (MH⁺) for | Intermediate 10 |
| | (3-chloro-2-methyl-7- | C₁₉H₂₁ClN₄O₄S | and methyl 2- |
| | oxo-1,7-dihydro-6H- | ¹H-NMR_δ: 1.74 (m, 1H); 1.79 (m, | bromo-1,3- |
| | pyrrolo[2,3-c]pyridin- | 2H); 2.45 (s, 3H); 2.60 (m, 1H); | thiazole-5- |
| | 6-yl)-3- | 3.25 (s, 3H); 3.35 (m, 1H); 3.84 (s, 3H); | carboxylate |
| | methoxypiperidin-1- | 4.30 (m, 1H); 4.55 (m, 1H); 5.39 (m, | |
| | yl]-1,3-thiazole-5- | 1H); 6.53 (d, 1H); 7.23 (d, 1H); 7.87 (s, | |
| | carboxylate | 1H); 11.36 (s, 1H) | |
| 10 | Ethyl 2-[4-(3-chloro-2- | MS (ES): 471 (MH⁺) for | Intermediate 6 |
| | methyl-7-oxo-1,7- | C₂₃H₂₃ClN₄O₃S | and ethyl 2- |
| | dihydro-6H- | ¹H-NMR_δ: 1.45 (t, 3H); 2.06 (m, 4H); | bromo-1,3- |
| | pyrrolo[2,3-c]pyridin- | 2.45 (s, 3H); 3.39 (m, 2H); 4.47 (q, | benzothiazole-7- |
| | 6-yl)piperidin-1-yl]- | 2H); 4.50 (m, 2H); 5.35 (m, 1H); | carboxylate |
| | 1,3-benzothiazole-7- | 6.56 (d, 1H); 6.98 (d, 1H); 7.39 (t, 1H); | (prepared as |
| | carboxylate | 7.79 (d, 1H); 7.82 (d, 1H); 11.30 (s, 1H) | described in US |
| | | | Pat 5,770,758) |
| 11 | Methyl 2-[4-(3-chloro- | MS (ES): 451 (MH⁺) for | Intermediate 6 |
| | 2-methyl-7-oxo-1,7- | C₂₀H₂₃ClN₄O₄S | and Intermediate |
| | dihydro-6H- | ¹H-NMR δ: 2.0 (m, 4H); 2.42 (s, 3H); | 25 |
| | pyrrolo[2,3-c]pyridin- | 3.30 (m, 2H); 3.49 (s, 3H); 3.82 (s, | |
| | 6-yl)piperidin-1-yl]-4- | 3H); 4.30 (m, 2H); 4.72 (s, 2H); | |
| | (methoxymethyl)-1,3- | 5.30 (m, 1H); 6.56 (d, 1H); 6.98 (d, 1H); | |
| | thiazole-5-carboxylate | 10.02 (s, 1H) | |
| 12 | Methyl 2-[4-(3-chloro- | MS (ES): 406 (MH⁺) for | Intermediate 6 |
| | 2-methyl-7-oxo-1,7- | C₁₈H₁₉ClN₄O₃S | and methyl 2- |
| | dihydro-6H- | | bromo-1,3- |
| | pyrrolo[2,3-c]pyridin- | | thiazole-5- |
| | 6-yl)piperidin-1-yl]- | | carboxylate |
| | 1,3-thiazole-5- | | |
| | carboxylate | | |
| 13 | Ethyl 2-[4-(3-chloro-2- | MS (ES): 421 (MH⁺) for | Intermediate 6 |
| | methyl-7-oxo-1,7- | C₁₉H₂₁ClN₄O₃S | and ethyl 2- |
| | dihydro-6H- | ¹H-NMR δ: 1.40 (t, 3H); 2.0 (m, 4H); | bromo-1,3- |
| | pyrrolo[2,3-c]pyridin- | 2.42 (s, 3H); 3.27 (m, 2H); 4.28 (m, | thiazole-4- |
| | 6-yl)piperidin-1-yl]- | 2H); 4.36 (q, 2H); 5.30 (m, 1H); | carboxylate |
| | 1,3-thiazole-4- | 6.58 (d, 1H); 6.98 (d, 1H); 7.49 (s, 1H); | |
| | carboxylate | 10.42 (s, 1H) | |
| 14 | Methyl 2-[(3S,4R)-4- | MS (ES): 425 (MH⁺) for | Intermediate 14 |
| | (3-chloro-2-methyl-7- | C₁₈H₁₈ClFN₄O₃S | and methyl 2- |
| | oxo-1,7-dihydro-6H- | ¹H-NMR_δ: 1.96 (m, 1H); 2.45 (s, 3H); | bromo-1,3- |
| | pyrrolo[2,3-c]pyridin- | 2.58 (m, 1H); 3.43 (m, 2H); 3.79 (s, | thiazole-5- |
| | 6-yl)-3-fluoropiperidin- | 3H); 4.56 (m, 2H); 4.87~5.04 (d, 1H); | carboxylate |
| | 1-yl]-1,3-thiazole-5- | 5.40 (m, br, 1H); 6.61 (d, 1H); 7.18 (d, | |
| | carboxylate | 1H); 7.89 (s, 1H); 10.14 (s, 1H) | |
| 15 | Methyl 2-chloro-6-[4- | MS (ES): 436 (MH⁺) for | Intermediate 6 |
| | (3-chloro-2-methyl-7- | C₁₉H₁₉Cl₂N₅O₃ | and methyl 2,4- |
| | oxo-1,7-dihydro-6H- | | dichloro- |
| | pyrrolo[2,3-c]pyridin- | | pyrimidine-4- |
| | 6-yl)piperidin-1- | | carboxylate |
| | yl]pyrimidine-4- | | |
| | carboxylate | | |

### Examples 16-23

Examples 16-23 were prepared by a procedure analogous to that described in Example 3 using the indicated starting materials.

| Ex | Compound | Data | SM |
|---|---|---|---|
| 16 | 2-[4-(3-Chloro-2-methyl-7- | MS (ES): 387 (MH⁺) for C₁₉H₁₉ClN₄O₃ | Example 8 |
| | oxo-1,7-dihydro-6H- | ¹H-NMR δ: 1.84 (m, 2H); 1.90 (m, 2H); | |
| | pyrrolo[2,3-c]pyridin-6- | 2.29 (s, 3H); 3.40 (m, 2H, overlapping | |
| | yl)piperidin-1-yl]isonicotinic | with H₂O); 4.52 (d, 2H); 5.14 (m, 1H); | |
| | acid | 6.36 (d, 1H); 7.07 (d, 1H); 7.26 (d, 1H); | |
| | | 7.36 (s, 1H); 8.24 (d, 1H); 12.17 (s, 1H) | |
| 17 | 2-Chloro-6-[4-(3-chloro-2- | MS (ES): 423 (MH⁺) for C₁₈H₁₇Cl₂N₅O₃ | Example |
| | methyl-7-oxo-1,7-dihydro-6H- | ¹H-NMR δ: 1.84 (m, 2H); 1.90 (m, 2H); | 15 |
| | pyrrolo[2,3-c]pyridin-6- | 2.29 (s, 3H); 3.18 (m, 2H); 3.40 (m, 2H, | |
| | yl)piperidin-1-yl]pyrimidine- | overlapping with H₂O); 5.22 (m, 1H); | |
| | 4-carboxylic acid | 6.36 (d, 1H); 7.32 (d, 1H); 7.38 (s, 1H); | |
| | | 12.23 (s, 1H); 13.70 (br, 1H) | |
| 18 | 2-[4-(3-Chloro-2-methyl-7- | MS (ES): 392.66 (MH⁺) for | Example |
| | oxo-1,7-dihydro-6H- | C₁₇H₁₇ClN₄O₃S | 12 |
| | pyrrolo[2,3-c]pyridin-6- | ¹H-NMR δ: 1.85 (m, 2H); 1.98 (m, 2H); | |
| | yl)piperidin-1-yl]-1,3-thiazole- | 2.29 (s, 3H); 3.35 (m, 2H); 4.11 (m, 2H); | |
| | 5-carboxylic acid | 5.14 (m, 1H); 6.36 (d, 1H); 7.14 (d, 1H); | |
| | | 7.79 (s, 1H); 12.25 (s, 1H); 12.66 (br, 1H) | |
| 19 | 2-[4-(3-Chloro-2-methyl-7- | MS (ES): 437 (MH⁺) for C₁₉H₂₁ClN₄O₄S | Example |
| | oxo-1,7-dihydro-6H- | ¹H-NMR δ: 1.83 (m, 2H); 2.0 (m, 2H); | 11 |
| | pyrrolo[2,3-c]pyridin-6- | 2.29 (s, 3H); 3.29 (m, 2H); 3.30 (s, 3H); | |
| | yl)piperidin-1-yl]-4- | 3.76 (s, 3H); 4.10 (m, 2H); 4.58 (s, 2H); | |
| | (methoxymethyl)-1,3-thiazole- | 5.12 (m, 1H); 6.38 (d, 1H); 7.30 (d, 1H); | |
| | 5-carboxylic acid | 12.17 (s, 1H) | |
| 20 | 2-[4-(3-Chloro-2-methyl-7- | MS (ES): 393 (MH⁺) for C₁₇H₁₇ClN₄O₃S | Example |
| | oxo-1,7-dihydro-6H- | ¹H-NMR δ: 1.91 (m, 4H); 2.29 (s, 3H); | 13 |
| | pyrrolo[2,3-c]pyridin-6- | 3.30 (m, 2H); 4.05 (m, 2H); 5.10 (m, 1H); | |
| | yl)piperidin-1-yl]-1,3-thiazole- | 6.37 (d, 1H); 7.31 (d, 1H); 7.65 (s, 1H); | |
| | 4-carboxylic acid | 12.21 (s, 1H) | |
| 21 | 2-[(3S,4R)-4-(3-Chloro-2- | MS (ES): 411 (MH⁺) for C₁₇H₁₆ClFN₄O₃S | Example |
| | methyl-7-oxo-1,7-dihydro-6H- | ¹H-NMR δ: 1.82 (m, 1H); 2.30 (s, 3H); | 14 |
| | pyrrolo[2,3-c]pyridin-6-yl)-3- | 2.73 (m, 1H); 3.60 (m, 2H); 4.14 (m, 1H); | |
| | fluoropiperidin-1-yl]-1,3- | 4.35 (m, 1H); 4.89~5.05 (d, 1H); 5.31 (m, | |
| | thiazole-5-carboxylic acid | 1H); 6.39 (d, 1H); 7.24 (d, 1H); 7.59 (s, | |
| | | 1H); 12.27 (s, 1H) | |
| 22 | 2-[3,4-cis-4-(3-Chloro-2- | MS (ES): 423 (MH⁺) for C₁₈H₁₉ClN₄O₄S | Example 9 |
| | methyl-7-oxo-1,7-dihydro-6H- | ¹H-NMR δ: 1.48 (m, 1H); 1.68 (m, 1H); | |
| | pyrrolo[2,3-c]pyridin-6-yl)-3- | 2.31 (s, 3H); 2.46 (m, 1H); 3.13 (s, 3H); | |
| | methoxypiperidin-1-yl]-1,3- | 3.61 (m, 2H); 4.13 (m, 1H); 4.36 (m, 1H); | |
| | thiazole-5-carboxylic acid | 5.20 (m, 1H); 6.33 (d, 1H); 7.23 (d, 1H); | |
| | | 7.75 (s, 1H); 12.23 (s, 1H) | |
| 23 | 2-[4-(3-Chloro-2-methyl-7- | MS (ES): 443 (MH⁺) for C₂₁H₁₉ClN₄O₃S | Example |
| | oxo-1,7-dihydro-6H- | ¹H-NMR δ: 1.88 (m, 2H); 2.0 (m, 2H); | 10 |
| | pyrrolo[2,3-c]pyridin-6- | 2.29 (s, 3H); 3.30 (m, 2H); 4.28 (m, 2H); | |
| | yl)piperidin-1-yl]-1,3- | 5.18 (m, 1H); 6.36 (d, 1H); 7.33 (d, 1H); | |
| | benzothiazole-7-carboxylic | 7.42 (t, 1H); 7.69 (d, 2H); 12.22 (s, 1H) | |
| | acid | | |

### Example 24

### Ethyl 2-[4-(3-chloro-2-methyl-8-oxo-4,5,6,8-tetrahydropyrrolo[2,3-c]azepin-7(1H)-yl)piperidin-1-yl]-1,3-thiazole-5-carboxylate

A solution of 85 mg (0.27 mmol) of 4-(3-chloro-2-methyl-8-oxo-4,5,6,8-tetrahydropyrrolo[2,3-c]azepin-7(1H)-yl)piperidinium chloride (Intermediate 17), (60 mg, 0.27 mmol) of ethyl 2-bromo-1,3-thiazole-5-carboxylate and 0.14 ml (0.8 mmol) Et₃N in 3 ml DMF was heated at 130° C. for 1 h in a microwave reactor. The mixture was poured into water. The solids were collected and dried in vacuo to give 65 mg of the title product.

MS (ES): (MH⁺) 437 for C₂₀H₂₅ClN₄O₃S.

### Examples 25-27

Examples 25-27 were prepared by a procedure analogous to that described in Example 24 using the indicated starting materials.

| Ex | Compound | Data | SM |
|---|---|---|---|
| 25 | Ethyl 2-[4-(3-chloro-2-methyl-8-oxo- | MS (ES): 437, | Intermediate 17 and |
| | 4,5,6,8-tetrahydropyrrolo[2,3-c]azepin- | 439 (MH⁺) for | ethyl 2-bromo-1,3- |
| | 7(1H)-yl)piperidin-1-yl]-1,3-thiazole-4- | C₂₀H₂₅ClN₄O₃S | thiazole-4- |
| | carboxylate | | carboxylate |
| 26 | Isopropyl 2-[4-(3-chloro-2-methyl-8- | MS (ES): 431, | Intermediate 17 and |
| | oxo-4,5,6,8-tetrahydropyrrolo[2,3- | 433 (MH⁺) for | isopropyl 2-fluoro |
| | c]azepin-7(1H)-yl)piperidin-1- | C₂₃H₂₉ClN₄O₃ | isonicotinate |
| | yl]isonicotinate | | |
| 27 | Isopropyl 2-[4-(3-chloro-2-methyl-8- | MS (ES): 465, | Intermediate 17 and |
| | oxo-4,5,6,8-tetrahydropyrrolo[2,3- | 467 (MH⁺) for | isopropyl 2-bromo- |
| | c]azepin-7(1H)-yl)piperidin-1-yl]-4- | C₂₂H₂₉ClN₄O₃S | 4-methyl-1,3- |
| | methyl-1,3-thiazole-5-carboxylate | | thiazole-5- |
| | | | carboxylate |
| 28 | Methyl 2-chloro-6-[4-(7-cyano-6- | | Intermediate 1 and |
| | methyl-2,4-dioxo-1,2,4,5-tetrahydro- | | methyl 2,4-dicholro- |
| | 3H-pyrrolo[3,2-d]pyrimidin-3- | | pyrimidine-6-carboxylate |
| | yl)piperidin-1-yl]pyrimidine-4- | | |
| | carboxylate | | |

### Example 29

### 2-[4-(3-Chloro-2-methyl-8-oxo-4,5,6,8-tetrahydropyrrolo[2,3-c]azepin-7(1H)-yl)piperidin-1-yl]-1,3-thiazole-5-carboxylic acid

A solution of 65 mg (0.15 mmol) of ethyl 2-[4-(3-chloro-2-methyl-8-oxo-4,5,6,8-tetrahydropyrrolo[2,3-c]azepin-7(1H)-yl)piperidin-1-yl]-1,3-thiazole-5-carboxylate (Example 24) and 0.3 ml of 2N LiOH in 3 ml MeOH was heated at 100° C. in a microwave reactor. The mixture was acidified with 0.65 ml 1N HCl. Solvent was removed and the residue was triturated with water to give a solid that was filtered, rinsed with water and dried in vacuo affording 53 mg of product.

MS (ES): 409, 411 (MH⁺) for C₁₈H₂₁ClN₄O₃S.

¹H-NMR δ: 1.5-2.0 (m, 6H); 2.15 (s, 3H); 3.0-3.3 (m, 6H); 4.1 (m, 2H); 4.7 (m, 1H); 7.8 (s, 1H); 11.3 (s, 1H); 12.6 (s, broad, 1H).

### Examples 30-32

Examples 30-32 were prepared by a procedure analogous to that described in Example 29 using the indicated starting materials.

| Ex | Compound | Data | SM |
|---|---|---|---|
| 30 | 2-[4-(3-Chloro-2-methyl-8- | MS (ES): 409, 411 (MH⁺) for | Example |
| | oxo-4,5,6,8- | C₁₈H₂₁ClN₄O₃S. | 25 |
| | tetrahydropyrrolo[2,3- | NMR δ: 1.5-2.0 (m, 6H); 2.15 (s, 3H); | |
| | c]azepin-7(1H)-yl)piperidin- | 3.0-3.3 (6H, m); 4.0 (m, 2H); 4.7 (m, | |
| | 1-yl]-1,3-thiazole-4- | 1H); 7.6 (s, 1H); 11.3 (s, 1H); 12.6 (s, | |
| | carboxylic acid | broad, 1H) | |
| 31 | 2-[4-(3-Chloro-2-methyl-8- | MS (ES): 403, 405 (MH⁺) for | Example |
| | oxo-4,5,6,8- | C₂₀H₂₃ClN₄O₃. | 26 |
| | tetrahydropyrrolo[2,3- | NMR δ: 1.6 (m, 4H); 1.8 (m, 2H); | |
| | c]azepin-7(1H)-yl)piperidin- | 2.15 (s, 3H); 3.0-3.3 (6H, m); 4.5 (m, 2H); | |
| | 1-yl]isonicotinic acid | 4.7 (m, 1H); 7.0 (d, 1H); 7.25 (s, 1H); | |
| | | 8.25 (d, 1H); 11.3 (s, 1H); 13.4 (s, broad, 1H) | |
| 32 | 2-[4-(3-Chloro-2-methyl-8- | MS (ES): 423, 425 (MH⁺) for | Example |
| | oxo-4,5,6,8- | C₁₉H₂₃ClN₄O₃S. | 27 |
| | tetrahydropyrrolo[2,3- | NMR δ: 1.5-2.0 (m, 6H); 2.15 (s, 3H); | |
| | c]azepin-7(1H)-yl)piperidin- | 2.4 (s, 3H); 3.1-3.4 (m, 6H); 4.0 (m, 2H); | |
| | 1-yl]-4-methyl-1,3-thiazole-5- | 4.7 (m, 1H); 7.6 (s, 1H); 11.3 (s, 1H); 12.6 (s, | |
| | carboxylic acid | broad, 1H) | |

### Preparation of Starting Materials

### Intermediate 1: 6-Methyl-2,4-dioxo-3-piperidin-4-yl-2,3,4,5-tetrahydro-1H-pyrrolo[3,2-d]pyrimidine-7-carbonitrile

Benzyl 4-(7-cyano-6-methyl-2,4-dioxo-1,2,4,5-tetrahydro-3H-pyrrolo[3,2-d]pyrimidin-3-yl)piperidine-1-carboxylate (Intermediate 2, 40 mg) was dissolved in methanol (10 ml), a catalytic amount of palladium (10%) on activated carbon was added and the mixture was flushed with nitrogen and hydrogen and stirred at room temperature under hydrogen overnight. The mixture was then filtered through diatomaceous earth and the filter cake was washed with methanol. The filtrate was concentrated to dryness and collected as the desired product (20 mg).

MS (ES): 274 (MH⁺) for C₁₃H₁₅N₅O₂

### Intermediate 2: Benzyl 4-(7-cyano-6-methyl-2,4-dioxo-1,2,4,5-tetrahydro-3H-pyrrolo[3,2-d]pyrimidin-3-yl)piperidine-1-carboxylate

Benzyl 4-[({[4-cyano-2-(methoxycarbonyl)-5-methyl-1H-pyrrol-3-yl]amino}carbonyl)amino]piperidine-1-carboxylate (Intermediate 3, 150 mg, 0.34 mmol) and potassium carbonate (47 mg, 0.34 mmol) were mixed with methanol (3 ml). The mixture was sealed in a microwave reaction tube and heated under microwave to 150° C. for 50 minutes. The mixture was cooled to room temperature and diluted with dichloromethane (10 ml), washed with water (6 ml), the organic layer was concentrated and purified by flash column chromatography and then eluted with 5% methanol in dichloromethane. The desired product was obtained as an off-white solid (45 mg).

MS (ES): 408 (MH⁺) for C₂₁H₂₁N₅O₄

¹H-NMR δ: 1.55 (m, 2H); 2.36 (s, 3H); 2.88 (m, 2H); 3.32 (m, 2H); 4.09 (m, 2H); 4.92 (m, 1H); 5.10 (s, 2H); 7.35 (m, 5H); 11.79 (br, 1H); 12.79 (br, 1H).

### Intermediate 3: Benzyl 4-[({[4-cyano-2-(methoxycarbonyl)-5-methyl-1H-pyrrol-3-yl]amino}carbonyl)amino]piperidine-1-carboxylate

Methyl-3-amino-4-cyano-5-methyl-1H-pyrrole-2-carboxylate (prepared as described in Heterocycles, 1989, 28(1), 51, 695 mg, 3.88 mmol), benzyl 4-isocyanatopiperidine-1-carboxylate (2000 mg, 7.68 mmol) and Et₃N (0.1 equivalence) were mixed in anhydrous acetonitrile (30 ml). The mixture was then refluxed for three days. After cooling to room temperature, the precipitate formed from the solution was collected by filtration and washed with dichloromethane, dried under high vacuum to give the desired product as a yellowish solid (1.6 g).

MS (ES): 440 (MH⁺) for C₂₂H₂₅N₅O₅

¹H-NMR (CDCl₃) δ: 1.40 (m, 2H); 1.95 (m, 2H); 2.45 (s, 3H); 2.94 (m, 2H); 3.86 (s, 3H); 3.88 (m, 1H); 4.14 (m, 2H); 5.11 (s, 2H); 7.36 (m, 5H); 7.89 (br, 1H); 8.64 (br, 1H); 9.60 (br, 1H).

### Intermediate 4: 3-(4-Bromophenyl)-6-methyl-2,4-dioxo-2,3,4,5-tetrahydro-1H-pyrrolo[3,2-d]pyrimidine-7-carbonitrile

Methyl-3-({[(4-bromophenyl)amino]carbonyl} amino)-4-cyano-5-methyl-1H-pyrrole-2-carboxylate (Intermediate 5) (600 mg, 1.59 mmol) and potassium carbonate (224 mg, 1.62 mmol) were mixed with methanol (15 ml). The mixture was sealed in a microwave reaction tube and heated under microwave to 160° C. for 50 minutes, cooled down to room temperature and diluted with dichloromethane (20 ml) and washed with water (20 ml). The organic layer was concentrated and purified by Gilson reverse phase (C-18) column chromatography (5%˜95% MeCN in H₂O, 0.1% TFA). The desired product was obtained as an off-white solid (200 mg).

MS (ES): 345 (MH⁺) for C₁₄H₉BrN₄O₂

¹H-NMR δ: 2.39 (s, 3H); 7.25 (d, 2H); 7.67 (d, 2H); 12.03 (s, 1H); 12.94 (s, 1H).

### Intermediate 5: Methyl 3-({[(4-bromophenyl)amino]carbonyl}amino)-4-cyano-5-methyl-1H-pyrrole-2-carboxylate

Methyl-3-amino-4-cyano-5-methyl-1H-pyrrole-2-carboxylate (prepared as described in Heterocycles, 1989, 28(1), 51, 300 mg, 1.68 mmol), 4-bromophenyl isocyanate (498 mg, 2.51 mmol) and triethylamine (0.2 ml) were mixed in anhydrous 1,2-dichloroethane (10 ml). The mixture was then refluxed overnight. After cooling down to room temperature, the desired product was obtained as a white solid (620 mg) by adding hexanes/dichloromethane and filtering the resulting precipitate.

MS (ES): 378 (MH⁺) for C₁₅H₁₃BrN₄O₃

### Intermediate 6: 3-Chloro-2-methyl-6-piperidin-4-yl-1,6-dihydro-7H-pyrrolo[2,3-c]pyridin-7-one

6-(1-Benzylpiperidin-4-yl)-3-chloro-2-methyl-1,6-dihydro-7H-pyrrolo[2,3-c]pyridin-7-one (Intermediate 7) (80 mg, 0.23 mmol) and 1,8-bis(dimethylamino)naphthalene (0.23 mmol) were dissolved in dry dichloroethane (10 ml), cooled down to 0° C., and 1-chloroethylchloroformate (48 mg, 0.34 mmol) was added. The reaction mixture was stirred for 15 minutes at room temperature and then heated up to 90° C. for 2 hours. After cooling down to room temperature, the solvent was removed under vacuum and the residue was dissolved in methanol (10 ml). The mixture was refluxed for 10 minutes and then concentrated to dryness. The resulting material was purified by column chromatography (10% methanol in dichloromethane) to give the desired product (42 mg).

MS (ES): 266 (MH⁺) for C₁₃H₁₆ClN₃O

¹H-NMR (CD₃OD) δ: 0.78 (m, 2H); 0.90 (m, 2H); 1.89 (m, 2H); 1.96 (s, 3H); 2.22 (m, 2H); 3.77 (m, 1H); 5.26 (d, 2H); 5.83 (d, 2H).

### Intermediate 7: 6-(1-Benzylpiperidin-4-yl)-3-chloro-2-methyl-1,6-dihydro-7H-pyrrolo[2,3-c]pyridin-7-one

N-(1-Benzylpiperidin-4-yl)-4-chloro-N-(1,3-dioxolan-2-ylmethyl)-5-methyl-1H-pyrrole-2-carboxamide (Intermediate 8) (400 mg, 0.96 mmol) was dissolved in methane sulfonic acid (10 ml) and stirred at 60° C. for two days. The reaction mixture was then cooled to room temperature, poured into a cold solution of sodium hydroxide (2M, 30 ml) and extracted with dichloromethane (3×50 ml). The combined organic layer was dried over anhydrous sodium sulfate and concentrated to an oil. Purification by column chromatography (10% methanol in dichloromethane) gave the desired product (80 mg).

MS (ES): 356 (MH⁺) for C₂₀H₂₂ClN₃O

¹H-NMR δ: 1.90 (m, 4H); 2.20 (m, 2H); 2.41 (s, 3H); 3.03 (m, 2H); 3.56 (s, 2H); 5.12 (m, 1H); 6.55 (d, 1H); 7.02 (d, 1H); 7.35 (m, 5H); 11.20 (s, 1H).

### Intermediate 8: N-(1-Benzylpiperidin-4-yl)-4-chloro-N-(1,3-dioxolan-2-ylmethyl)-5-methyl-1H-pyrrole-2-carboxamide

4-Chloro-5-methyl-1H-pyrrole-2-carboxylic acid (Intermediate 23, 319 mg, 2 mmol), 1-(3-dimethylaminopropyl)-3-ethylcarbodiimide hydrochloride (460 mg, 2.4 mmol), N-methylmorpholine (488 mg, 4.8 mmol) and 1-hydroxybenzotriazole (324 mg, 2.4 mmol) were mixed in anhydrous dichloromethane (20 ml) at 0° C. and stirred for 10 minutes. 1-Benzyl-N-(1,3-dioxolan-2-ylmethyl)piperidin-4-amine (Intermediate 9) (552 mg, 2 mmol) was added and the reaction mixture was stirred at room temperature for 12 hours. The reaction mixture was diluted with dichloromethane (20 ml), washed with saturated aqueous ammonium chloride solution and brine, concentrated and purified by column chromatography (5% methanol in dichloromethane) to give the title product (485 mg).

MS (ES): 418 (MH⁺) for C₂₂H₂₈ClN₃O₃

¹H-NMR (CDCl₃) δ: 1.75 (m, 2H); 1.95 (m, 2H); 2.05 (m, 2H); 2.24 (s, 3H); 2.98 (m, 2H); 3.52 (s, 2H); 3.64 (m, 2H); 3.86 (m, 2H); 4.00 (m, 2H); 4.30 (m, 1H); 5.08 (t, 1H); 6.47 (s, br, 1H); 7.22-7.31 (m, 5H); 9.41 (br s, 1H).

### Intermediate 9: 1-Benzyl-N-(1,3-dioxolan-2-ylmethyl)piperidin-4-amine

(1,3-Dioxolan-2-ylmethyl)amine (1.6 g, 15.5 mmol) and 1-benzylpiperidin-4-one (2.94 g, 15.5 mmol) were dissolved in ethylene-dichloride (30 ml), two drops of acidic acid were added and the mixture was stirred at room temperature for 5 minutes, followed by the addition of sodium triacetoxy borohydride (4.93 g, 23.3 mmol). The resulting reaction mixture was stirred at room temperature overnight, washed with saturated aqueous sodium bicarbonate (20 ml) and brine, dried over anhydrous magnesium sulfate, concentrated and purified by column chromatography (5% methanol in dichloromethane). The desired product was obtained as an oil (4.4 g).

GCMS: 276 (MW) for C₁₆H₂₄N₂O₂

¹H-NMR (CDCl₃) δ: 1.41 (m, 2H); 1.85 (m, 2H); 2.01 (m, 2H); 2.48 (m, 1H); 2.82 (d, 2H); 2.86 (m, 2H); 3.49 (s, 2H); 3.88 (m, 2H); 3.99 (m, 2H); 4.97 (t, 1H); 7.22-7.31 (m, 5H).

### Intermediate 10: 3-Chloro-6-[(3,4)-cis-3-methoxypiperidin-4-yl]-2-methyl-1,6-dihydro-7H-pyrrolo[2,3-c]pyridin-7-one

The title compound was synthesized from ethyl (3,4)-cis-4-(3-chloro-2-methyl-7-oxo-1,7-dihydro-6H-pyrrolo[2,3-c]pyridin-6-yl)-3-methoxypiperidine-1-carboxylate (Intermediate 11) by treatment with 1 eq. of trimethyl silyl iodide at reflux for 2 h. Quantitative yield.

MS (ES): 296 (MH⁺) for C₁₄H₁₈ClN₃O₂

¹H-NMR (CDCl₃) δ: 1.52 (m, 2H); 2.36 (s, 3H); 2.75 (m, 2H); 3.11 (s, 3H); 3.25 (m, 2H); 3.39 (m, 1H); 5.19 (m, 1H); 6.44 (d, 1H); 7.22 (d, 1H); 11.63 (br s, 1H).

### Intermediate 11: Ethyl (3,4)-cis-4-(3-chloro-2-methyl-7-oxo-1,7-dihydro-6H-pyrrolo[2,3-c]pyridin-6-yl)-3-methoxypiperidine-1-carboxylate

Intermediate 11 was synthesized by a procedure analogous to the procedure described for Intermediate 7 but starting with ethyl (3,4)-cis-4-[[(4-chloro-5-methyl-1H-pyrrol-2-yl)carbonyl](1,3-dioxolan-2-ylmethyl)amino]-3-methoxypiperidine-1-carboxylate (Intermediate 12).

MS (ES): 368 (MH⁺) for C₁₇H₂₂ClN₃O₄

¹H-NMR (CDCl₃) δ: 1.30 (t, 3H); 1.63 (m, 1H); 1.77 (m, 2H); 2.44 (s, 3H); 2.94 (m, 2H); 3.25 (s, br, 3H); 3.54 (m, 1H); 4.19 (m, 2H); 4.24 (m, 1H); 5.30 (m, 1H); 6.52 (d, 1H); 7.26 (d, 1H); 11.44 (s, br, 1H).

### Intermediate 12: Ethyl (3,4)-cis-4-[[(4-chloro-5-methyl-1H-pyrrol-2-yl)carbonyl] (1,3-dioxolan-2-ylmethyl)amino]-3-methoxypiperidine-1-carboxylate

Intermediate 12 was synthesized by a procedure analogous to the procedure described for Intermediate 8 but starting with ethyl (3,4)-cis-4-[(1,3-dioxolan-2-ylmethyl)amino]-3-methoxypiperidine-1-carboxylate (Intermediate 13).

MS (ES): 430 (MH⁺) for C₁₉H₂₈ClN₃O₆

¹H-NMR (CDCl₃) δ: 1.27 (m, 3H); 1.65 (m, 2H); 1.89 (m, 1H); 2.25 (s, 3H); 2.81 (m, 2H); 3.20 (m, 1H); 3.30 (s, 3H); 3.54 (m, 2H); 3.86 (m, 2H); 4.00 (m, 2H); 4.16 (m, 2H); 4.37 (m, 1H); 4.61 (m, 11H); 5.08 (m, 11H); 6.58 (s, br, 1H); 9.99 (s, br, 1H).

### Intermediate 13: Ethyl (3,4)-cis-4-[(1,3-dioxolan-2-ylmethyl)amino]-3-methoxypiperidine-1-carboxylate

Intermediate 13 was synthesized by a procedure analogous to the procedure described for Intermediate 16 but starting with 2-(bromomethyl)-1,3-dioxolane and cis(±) ethyl 4-amino-3-methoxypiperidine-1-carboxylate hydrochloride salt (Intermediate 26).

MS (ES): 289 (MH⁺) for C₁₃H₂₄N₂O₅

¹H-NMR (CDCl₃) δ: 1.25 (t, 3H); 1.62 (m, 4H); 2.74 (m, 1H); 2.79 (m, 2H); 2.91 (m, 2H); 3.39 (s, 3H); 3.88 (m, 2H); 3.99 (m, 2H); 4.13 (m, 2H); 4.15 (m, 1H); 4.97 (m, 1H).

### Intermediate 14: 3-Chloro-6-[(3S,4R)-3-fluoropiperidin-4-yl]-2-methyl-1,6-dihydro-7H-pyrrolo[2,3-c]pyridin-7-one

The title compound was synthesized from tert-butyl (3S,4R)-4-[[(4-chloro-5-methyl-1H-pyrrol-2-yl)carbonyl](1,3-dioxolan-2-ylmethyl)amino]-3-fluoropiperidine-1-carboxylate (Intermediate 15) by the procedure of Intermediate 7. The resulting precipitate was collected by filtration. Quantitative yield.

MS (ES): 284 (MH⁺) for C₁₃H₁₅CFN₃O.

### Intermediate 15: tert-Butyl (3S,4R)-4-[[(4-chloro-5-methyl-1H-pyrrol-2-yl)carbonyl] (1,3-dioxolan-2-ylmethyl)amino]-3-fluoropiperidine-1-carboxylate

Intermediate 15 was synthesized by a procedure analogous to the procedure described for Intermediate 8 but starting with tert-butyl (3S,4R)-4-[(1,3-dioxolan-2-ylmethyl)amino]-3-fluoropiperidine-1-carboxylate (Intermediate 16).

MS (ES): 446 (MH⁺) for C₂₀H₂₉ClFN₃O₅

¹H-NMR (CDCl₃) δ: 1.47 (s, 9H); 1.78 (m, 2H); 1.95 (m, 1H); 2.24 (s, 3H); 2.88 (m, 2H); 3.71 (m, 1H); 3.86 (m, 2H); 4.00 (m, 2H); 4.39 (m, 2H); 4.60-4.93 (m, 2H); 5.08 (m, 1H); 6.62 (s, br, 1H); 9.96 (br s, 1H).

### Intermediate 16: tert-Butyl (3S,4R)-4-[(1,3-dioxolan-2-ylmethyl)amino]-3-fluoropiperidine-1-carboxylate

tert-butyl (3S,4R)-4-amino-3-fluoropiperidine-1-carboxylate (Intermediate 29) (820 mg, 3.78 mmol), 2-bromomethyl-1,3-dioxolane (630 mg, 3.78 mmol) and potassium carbonate (784 mg, 5.67 mmol) were mixed in anhydrous acetonitrile (15 ml), refluxed for 5 days, cooled down to room temperature and diluted with water (50 ml). The resulting mixture was extracted with EtOAc (4×30 ml), the organic layer was washed with brine and dried over sodium sulfate, concentrated and purified by flash column chromatography (5% MeOH in DCM) to give the desired product (730 mg).

MS (ES): 305 (MH⁺) for C₁₄H₂₅FN₂O₄

¹H-NMR (CDCl₃) δ: 1.45 (s, 9H); 1.78 (m, 2H); 2.76 (m, 2H); 2.88 (m, 2H); 3.86 (m, 2H); 4.00 (m, 2H); 4.36 (m, 2H); 4.55-4.81 (m, 2H); 4.97 (t, 1H).

### Intermediate 17: 4-(3-Chloro-2-methyl-8-oxo-4,5,6,8-tetrahydropyrrolo[2,3-c]azepin-7(11H)-yl)piperidinium chloride

A solution of 4M HCl (4 ml, 16 mmol) in dioxane was added to a solution of 420 mg (1.1 mmol) of tert-butyl 4-(3-chloro-2-methyl-8-oxo-4,5,6,8-tetrahydropyrrolo[2,3-c]azepin-7(1H)-yl)piperidine-1-carboxylate (Intermediate 18) in 10 ml dioxane. The mixture was stirred at room temperature for 3 days. The solvent was removed and the resulting solid was dried in vacuo to give 345 mg of product.

MS (ES): 359 (MH⁺) for C₁₄H₂₀ClN₄O₃S—HCl;

NMR δ: 1.6-1.75 (m, 2H); 1.9 (m, 3H); 2.15 (s, 3H); 2.6 (t, 2H); 3.0 (m, 2H); 3.2-3.4 (m, 5H); 4.7 (m, 1H); 7.8 (s, broad, 2H); 11.3 (s, 1H).

### Intermediate 18: tert-Butyl 4-(3-chloro-2-methyl-8-oxo-4,5,6,8-tetrahydropyrrolo[2,3-c]azepin-7(11H)-yl)piperidine-1-carboxylate

A solution of 1.15 g (2.7 mmol) of tert-butyl 4-({3-[4-chloro-2-(ethoxycarbonyl)-5-methyl-1H-pyrrol-3-yl]prop-2-yn-1-yl} amino)piperidine-1-carboxylate (Intermediate 19) with 230 mg platinum oxide was hydrogenated at atmospheric pressure overnight. The mixture was filtered through diatomaceous earth, rinsing through with EtOAc. The solvent was removed and the residue was dissolved in 6 ml MeOH. A solution of 2N lithium hydroxide (2.7 ml, 5.4 mmol) was added and the mixture was heated at 100° C. for 1 h. The mixture was diluted with MeOH and 4 ml of 1N HCl was added. The solvent was removed and the residue was dissolved in additional MeOH. The solvent was again removed and the residue was dissolved in THF. Solvent was removed and the residue was dissolved in 30 ml DMF. Et₃N (0.75 ml, 5.4 mmol) and 1.0 g (2.7 mmol) of HATU was added. The mixture was stirred at room temperature overnight. The solvent was removed, and the residue was dissolved in EtOAc. The mixture was washed with water and brine, dried over magnesium sulphate and the solvent removed. The residue was chromatographed on silica gel (100% CH₂Cl₂ with gradient elution to 60% EtOAc in CH₂Cl₂) to afford 420 mg of product as a solid.

MS (ES): 382 (MH⁺) for C₁₉H₂₈ClN₃O₃;

NMR δ: 1.4 (s, 9H); 1.5 (m, 4H0, 1.85 (m, 2H); 2.1 (s, 3H); 2.6 (t, 2H); 2.8 (m, 2H); 3.2 (m, 2H); 4.0 (m, 2H); 4.6 (m, 1H); 11.3 (s, 1H).

### Intermediate 19: tert-Butyl 4-({3-[4-chloro-2-(ethoxycarbonyl)-5-methyl-1H-pyrrol-3-yl]prop-2-yn-1-yl}amino)piperidine-1-carboxylate

A solution of 2.5 g (8 mmol) of 1-tert-butyl 2-ethyl 4-chloro-3-iodo-5-methyl-1H-pyrrole-1,2-dicarboxylate (Intermediate 21), 2.2 g (9.6 mmol) of tert-butyl 4-(prop-2-yn-1-ylamino)piperidine-1-carboxylate (Intermediate 20), 360 mg (0.56 mmol) of bis(diphenylphosphino)dichloropalladium, 98 mg copper iodide and 529 mg triphenylphosphine in 30 ml diethylamine was heated under N₂ at 60° C. overnight. The solvent was removed and the residue was diluted with CH₂Cl₂ and washed with water and brine. Drying (MgSO₄) and removal of solvent gave an oil that was chromatographed on silica gel (100% CH₂Cl₂ with gradient elution to 100% EtOAc) to afford 1.15 g of product as a solid.

MS (ES): 424, 426 (MH⁺) for C₂₁H₃₀ClN₃O₄.

NMR δ: 1.2 (m, 2H); 1.3 (t, 3H); 1.4 (s, 9H); 1.8 (m, 2H); 2.2 (s, 3H); 2.8-3.0 (m, 4H); 3.6 (s, 1H); 3.8 (m, 1H); 4.2 (q, 2H); 12.2 (s, 1H).

### Intermediate 20: tert-Butyl 4-(prop-2-yn-1-ylamino)piperidine-1-carboxylate

A solution of 6.6 g (33 mmol) of tert-butyl 4-oxopiperidine-1-carboxylate, 2.4 ml (37.5 mmol) propargylamine and 10.5 g (49.5 mmol) sodium triacetoxyborohydride in 100 ml CH₂Cl₂ was stirred at room temperature overnight. The mixture was quenched with 1N HCl and then basified with saturated aqueous sodium carbonate before being extracted twice with CH₂Cl₂. The combined extracts were washed with brine, dried (MgSO₄) and concentrated to afford 7.1 g of product as a white solid.

NMR δ: 1.0-1.2 (m, 2H); 1.4 (s, 9H); 1.6-1.8 (m, 2H); 2.6-2.9 (m, 2H); 3.0 (t, 1H); 3.3 (d, 2H); 3.7-3.9 (m, 2H).

### Intermediate 21: 1-tert-Butyl 2-ethyl 4-chloro-3-iodo-5-methyl-1H-pyrrole-1,2-dicarboxylate

A solution of 5.6 g (18 mmol) of ethyl 4-chloro-3-iodo-5-methyl-1H-pyrrole-2-carboxylate (Intermediate 22), 5.1 g (23.4 mmol) of di-t-butyldicarbonate and 4.5 ml (4.5 mmol) of Et₃N in 40 ml THF was heated at reflux overnight. The solution was diluted with saturated aqueous ammonium chloride and EtOAc. The EtOAc was separated and washed with brine. The combined aqueous layers were re-extracted with EtOAc, which was washed with brine. The combined EtOAc extracts were dried (MgSO₄), dried and concentrated. The residue was chromatographed on silica gel (100% hexanes with gradient elution to 100% CH₂Cl₂) to afford 6.8 g of the product as an oil that slowly solidified.

NMR δ: 1.3 (t, 3H) 1.5 (s, 9H); 2.4 (s, 31H); 4.3 (q, 2H).

### Intermediate 22: Ethyl 4-chloro-3-iodo-5-methyl-1H-pyrrole-2-carboxylate

Iodine (5.4 g, 21 mmol) was added portion wise to a solution of 3.5 g (19 mmol) of ethyl 4-chloro-5-methyl-1H-pyrrole-2-carboxylate (Intermediate 24) and 3.5 g (47 mmol) of 75% potassium hydroxide in 20 ml DMF. After stirring for two hours, the mixture was acidified with 1N HCl and diluted with water. Insoluble solids were filtered, washed with water and dried in vacuo overnight affording 5.5 g of product.

MS (ES): 314 289 (MH⁺) for C₈H₉ClINO₂

NMR δ: 1.45 (t, 3H); 2.4 (s, 3H); 4.4 (q, 2H); 12.5 (s, 1H).

### Intermediate 23: 4-Chloro-5-methyl-1H-pyrrole-2-carboxylic acid

Lithium hydroxide (2 M, 4 ml) was warmed to 50° C. and a solution of ethyl 4-chloro-5-methyl-1H-pyrrole-2-carboxylate (Intermediate 24; 0.30 g, 1.60 mmol) in MeOH was added to it. The reaction was heated to 80° C. and stirred for two hours. The MeOH was removed and the aqueous solution was cooled to 0° C. and acidified with 30% HCl. The precipitated product (0.23 g, 92%) was filtered and dried.

MS (ES): 160 (M+1) for C₆H₆ClNO₂

NMR (CDCl₃): 2.25 (s, 3H); 6.85 (s, 1H); 8.98 (brs, 1H).

### Intermediate 24: Ethyl 4-chloro-5-methyl-1H-pyrrole-2-carboxylate

N-Chlorosuccinimide (0.67 g, 5.08 mmol) was added to a solution of ethyl 5-methyl-1H-pyrrole-2-carboxylate (0.65 g, 4.23 mmol) in chloroform (20 ml). The reaction was warmed to 40° C. and stirred for 4 h, then poured to a beaker containing 2 N NaOH (20 ml) at 0° C. The layers were separated and the aqueous layer was extracted with chloroform (×3). The combined organic extracts were dried over magnesium sulfate and concentrated. The resultant off-white solid was purified by flash chromatography (hexanes/EtOAc, 16:1) to give the title product as a white solid (0.3 g, 38%).

MS (ES): 188 (M+1) for C₈H₁₀ClNO₂

NMR (CDCl₃): 1.34 (t, 3H); 2.27 (s, 3H); 4.30 (q, 2H); 6.76 (s, 1H); 9.07 (brs, 1H)

### Intermediate 25: Methyl 2-chloro-4-(methoxymethyl)-1,3-thiazole-5-carboxylate

tert-Butyl nitrite (2.2 ml, 18.6 mmol) and cuprous chloride (1.5 g) were suspended in anhydrous CH₃CN (100 ml). Methyl 2-amino-4-(methoxymethyl)-1,3-thiazole-5-carboxylate (2.5 g) (prepared as described in Kennedy, Alan R. et al. Acta Crystallographica, Section C: Crystal Structure Communications 1999, C55 (7) 2) was added in one portion. The mixture was stirred at room temperature for 2 h and the temperature was raised to 70° C. for 1 h. The mixture was cooled to room temperature and filtered. The filtrate was poured into 6 N HCl, extracted with EtOAc, dried with MgSO₄ and concentrated to a black oil. Flash purification on silica gel with gradient elution (hexane to EtOAc) yielded product as a yellow liquid (0.82 g).

NMR: 3.31 (s, 3H); 3.85 (s, 3H); 4.71 (s, 2H).

### Intermediate 26: cis(±) Ethyl 4-amino-3-methoxypiperidine-1-carboxylate hydrochloride salt

The title compound can be prepared as described in Lee, C. et al. Synth. Comm. 2001, 31(7), 10881-10890 and/or WO 94/12494 or by the following procedure;

To a stirred solution of 1-piperidinecarboxylic acid, cis(±)3-methoxy-4-[benzylamino]-, ethyl ester (Drug Development Research 1986, 8, 225-232; 36.45 g, 125 mmol) and 10% palladium on activated carbon (50% wet; approximately 4 g) in methanol (250 mL), at room temperature and under an atmosphere of N₂, was added ammonium formate (31.50 g, 500 mmol) as a solid. The temperature was increased to 70° C.; the reaction was stirred overnight at this temperature, under an atmosphere of N₂. Complete conversion was suggested by TLC (6% methanol in ethyl acetate; Rf˜0.06 in a solution of 15% methanol and 30% acetone in DCM) in the morning. The reaction mixture was filtered through diatomaceous earth and concentrated under vacuum. To the residue was added approximately 50 mL water; from this mixture was extracted the crude product with a solution of 3% methanol in chloroform (4×300 mL). Organic layers were combined, dried over magnesium sulfate, and concentrated. Obtained 24.18 g (96%) of an off-white solid.

MS (ES) MH⁺: 202 for C₉H₁₈N₂O₃

### Intermediate 27: cis(±) tert-Butyl 4-(benzylamino)-3-fluoropiperidine-1-carboxylate

The title compound was prepared as described in Monique B. van Neil et al. J. Med. Chem., 1999, 42, 2087-2104 and the references therein.

NMR (CDCl₃): 1.40 (s, 9H); 1.88 (m, 2H); 3.01 (m, 2H); 3.55 (m, 2H); 3.77 (m, 1H); 4.66 (d, 1H).

### Intermediate 28: tert-Butyl (3S,4R)-4-(benzylamino)-3-fluoropiperidine-1-carboxylate and tert-Butyl (3R,4S)-4-(benzylamino)-3-fluoropiperidine-1-carboxylate

cis(±) tert-Butyl-4-(benzylamino)-3-fluoropiperidine-1-carboxylate (Intermediate 27) (2.2 g) was separated into the title compounds using chiral HPLC over a Chiralpak AD column (eluent: hexanes/MeOH/EtOH; 90/2.5/2.5; 0.1% diethylamine). The fractions corresponding to the first chromatographic peak (tert-Butyl (3S,4R)-4-amino-3-fluoropiperidine-1-carboxylate) were collected and evaporated yielding the title compound as a white solid (942 mg). The fractions corresponding to the second chromatographic peak (tert-Butyl (3R,4S)-4-amino-3-fluoropiperidine-1-carboxylate) were collected and evaporated yielding the title compound as a white solid (980 mg).

NMR (CDCl₃): 1.40 (s, 9H); 1.88 (m, 2H); 2.05 (m, 2H); 3.01 (m, 2H); 3.55 (m, 2H); 3.77 (m, 1H); 4.66 (d, 1H); 7.55 (m, 5H).

### Intermediate 29: tert-Butyl (3S,4R)-4-amino-3-fluoropiperidine-1-carboxylate

tert-Butyl (3S,4R)-4-(benzylamino)-3-fluoropiperidine-1-carboxylate (Intermediate 28; 711 mg), ammonium formate (582 mg), and 10% Pd/C (200 mg) in MeOH (10 ml) was heated to 50° C. for 1 h. The reaction mixture was cooled to room temperature, filtered through diatomaceous earth and concentrated under reduced pressure to give the title compound (503 mg, quantitative).

NMR (CDCl₃): 1.40 (s, 9H); 1.88 (m, 2H); 3.01 (m, 2H); 3.55 (m, 2H); 3.77 (m, 1H); 4.66 (d, 1H).

1. A compound of formula (I): 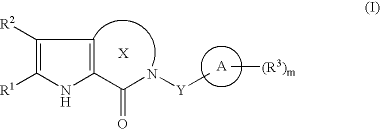
wherein:
R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)a wherein a is 0 to 2 and C₃₋₆cycloalkyl; wherein R¹ may be optionally substituted on carbon by one or more halo or cyclopropyl;
R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)a wherein a is 0 to 2 and C₃₋₆cycloalkyl; wherein R² may be optionally substituted on carbon by one or more halo or C₃₋₆cycloalkyl;
R³ represents a substituent on carbon and is selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂-amino, C₁₋₄alkanoylamino, N—(C₁₋₄alkyl)carbamoyl, N,N—(C₁₋₄alkyl)₂-carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)a wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R⁴— or heterocyclyl-R⁵—; wherein R³ may be optionally substituted on carbon by one or more R⁶; and wherein if said heterocyclyl contains an —NH— moiety that nitrogen may be optionally substituted by a group selected from R⁷;
R⁶ is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)₃ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹³— or heterocyclyl-R¹⁴—; wherein R⁶ may be optionally substituted on carbon by one or more R¹⁵; and wherein if said heterocyclyl contains an —NH— moiety that nitrogen may be optionally substituted by a group selected from R¹⁶;
R⁴, R⁵, R¹³ and R¹⁴ are independently selected from a direct bond, —O—, —N(R⁸)—, —C(O)—, —N(R⁹)C(O)—, —C(O)N(R¹⁰)—, —S(O)ₚ—, —SO₂N(R¹¹)— or —N(R¹²)SO₂—; wherein R⁸, R⁹, R¹⁰, R¹¹ and R¹² are independently selected from hydrogen or C₁₋₄alkyl and p is 0-2;
R¹⁵ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Ring X is a heterocyclic ring selected from X¹, X², X³ and X⁴;
X¹ is 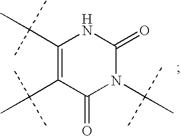
X² is 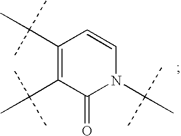
X³ is 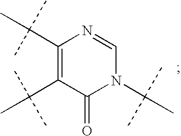
X⁴ is 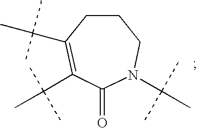
Y is selected from phenyl, azetidinyl, piperidinyl and pyrrolidinyl; wherein the N of said azetidinyl, piperidinyl and pyrrolidinyl ring is directly attached to Ring A; and further wherein Y may be optionally substituted on carbon by one or two halo, C₁₋₄alkyl or C₁₋₄alkoxy;
Ring A is carbocyclyl or heterocyclyl; wherein if said heterocyclyl contains an —NH— moiety that nitrogen may be optionally substituted by a group selected from R¹⁷;
m is 0-4; wherein the values of R³ may be the same or different;
R⁷, R¹⁶ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;
or a pharmaceutically acceptable salt thereof.

2. A compound of formula (I), or a pharmaceutically acceptable salt thereof, as claimed in claim 1, wherein R¹ is C₁₋₄alkyl.

3. A compound of formula (I), or a pharmaceutically acceptable salt thereof, as claimed in claim 2, wherein R² is halo or cyano.

4. A compound of formula (I), or a pharmaceutically acceptable salt thereof, as claimed in claim 3, wherein Ring X is a heterocyclic ring selected from X¹.

5. A compound of formula (I), or a pharmaceutically acceptable salt thereof, as claimed in claim 3, wherein Ring X is a heterocyclic ring selected from X².

6. A compound of formula (I), or a pharmaceutically acceptable salt thereof, as claimed in claim 3, wherein Ring X is a heterocyclic ring selected from X³.

7. A compound of formula (I), or a pharmaceutically acceptable salt thereof, as claimed in claim 3, wherein Ring X is a heterocyclic ring selected from X⁴.

8. A compound of formula (I), or a pharmaceutically acceptable salt thereof, as claimed in claim 4, wherein Y is selected from phenyl and piperidinyl; wherein the N of said piperidinyl ring is directly attached to Ring A; and further wherein Y may be optionally substituted on carbon by one halo or C₁₋₄alkoxy.

9. A compound of formula (I), or a pharmaceutically acceptable salt thereof, as claimed in claim 8, wherein Ring A is phenyl, thiazolyl, benzothiazolyl, pyrimidinyl or pyridinyl.

10. A compound of formula (I), or a pharmaceutically acceptable salt thereof, as claimed in claim 9, wherein R³ represents a substituent on carbon and is selected from halo, carboxy, C₁₋₄alkyl, C₁₋₄alkoxy or C₁₋₄alkoxycarbonyl; wherein R³ may be optionally substituted on carbon by one or more R⁶; wherein R⁶ is selected from C₁₋₄alkoxy.

11. A compound of formula (I), or a pharmaceutically acceptable salt thereof, as claimed in claim 10, wherein m is 1 or 2; wherein the values of R³ may be the same or different.

12. A compound of formula (I): 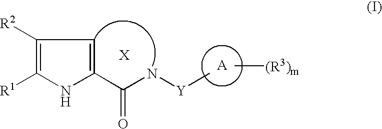
wherein:
R¹ is methyl;
R² is chloro or cyano;
Ring X is a heterocyclic ring selected from X¹, X² or X⁴;
X¹ is 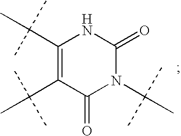
X² is 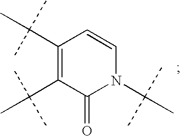
X⁴ is 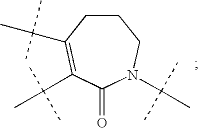
Y is selected from phenyl, 3-fluoropiperidinyl and 3-methoxypiperidinyl; wherein the N of said piperidinyl ring is directly attached to Ring A;
Ring A is phenyl, thiazol-2-yl, benzothiazol-2-yl, pyrimidin-4-yl or pyridin-2-yl;
R³ represents a substituent on carbon and is selected from fluoro, chloro, carboxy, methyl, methoxy, methoxycarbonyl, methoxymethyl, ethoxycarbonyl or isopropoxycarbonyl; and
m is 1 or 2; wherein the values of R³ may be the same or different;
or a pharmaceutically acceptable salt thereof.

13. A compound of formula (I): 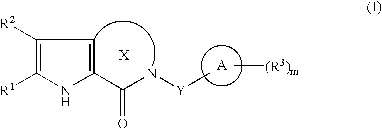
selected from:
methyl 2-[4-(7-cyano-6-methyl-2,4-dioxo-1,2,4,5-tetrahydro-3H-pyrrolo[3,2-d]pyrimidin-3-yl)piperidin-1-yl]-1,3-thiazole-5-carboxylate;
ethyl 2-[4-(7-cyano-6-methyl-2,4-dioxo-1,2,4,5-tetrahydro-3H-pyrrolo[3,2-d]pyrimidin-3-yl)piperidin-1-yl]-1,3-benzothiazole-7-carboxylate;
2-[4-(7-cyano-6-methyl-2,4-dioxo-1,2,4,5-tetrahydro-3H-pyrrolo[3,2-d]pyrimidin-3-yl)piperidin-1-yl]-1,3-thiazole-5-carboxylic acid;
2-[4-(7-cyano-6-methyl-2,4-dioxo-1,2,4,5-tetrahydro-3H-pyrrolo[3,2-d]pyrimidin-3-yl)piperidin-1-yl]-1,3-benzothiazole-7-carboxylic acid;
6-[4-(7-cyano-6-methyl-2,4-dioxo-1,2,4,5-tetrahydro-3H-pyrrolo[3,2-d]pyrimidin-3-yl)piperidin-1-yl]-2-methoxypyrimidine-4-carboxylic acid;
4′-(7-cyano-6-methyl-2,4-dioxo-1,2,4,5-tetrahydro-3H-pyrrolo[3,2-d]pyrimidin-3-yl)biphenyl-3-carboxylic acid;
4′-(7-cyano-6-methyl-2,4-dioxo-1,2,4,5-tetrahydro-3H-pyrrolo[3,2-d]pyrimidin-3-yl)-4-fluorobiphenyl-3-carboxylic acid;
ethyl 2-[4-(3-chloro-2-methyl-7-oxo-1,7-dihydro-6H-pyrrolo[2,3-c]pyridin-6-yl)piperidin-1-yl]isonicotinate;
methyl 2-[(3,4)-cis-4-(3-chloro-2-methyl-7-oxo-1,7-dihydro-6H-pyrrolo[2,3-c]pyridin-6-yl)-3-methoxypiperidin-1-yl]-1,3-thiazole-5-carboxylate;
ethyl 2-[4-(3-chloro-2-methyl-7-oxo-1,7-dihydro-6H-pyrrolo[2,3-c]pyridin-6-yl)piperidin-1-yl]-1,3-benzothiazole-7-carboxylate;
methyl 2-[4-(3-chloro-2-methyl-7-oxo-1,7-dihydro-6H-pyrrolo[2,3-c]pyridin-6-yl)piperidin-1-yl]-4-(methoxymethyl)-1,3-thiazole-5-carboxylate;
methyl 2-[4-(3-chloro-2-methyl-7-oxo-1,7-dihydro-6H-pyrrolo[2,3-c]pyridin-6-yl)piperidin-1-yl]-1,3-thiazole-5-carboxylate;
ethyl 2-[4-(3-chloro-2-methyl-7-oxo-1,7-dihydro-6H-pyrrolo[2,3-c]pyridin-6-yl)piperidin-1-yl]-1,3-thiazole-4-carboxylate;
methyl 2-[(3S,4R)-4-(3-chloro-2-methyl-7-oxo-1,7-dihydro-6H-pyrrolo[2,3-c]pyridin-6-yl)-3-fluoropiperidin-1-yl]-1,3-thiazole-5-carboxylate;
methyl 2-chloro-6-[4-(3-chloro-2-methyl-7-oxo-1,7-dihydro-6H-pyrrolo[2,3-c]pyridin-6-yl)piperidin-1-yl]pyrimidine-4-carboxylate;
2-[4-(3-chloro-2-methyl-7-oxo-1,7-dihydro-6H-pyrrolo[2,3-c]pyridin-6-yl)piperidin-1-yl]isonicotinic acid;
2-chloro-6-[4-(3-chloro-2-methyl-7-oxo-1,7-dihydro-6H-pyrrolo[2,3-c]pyridin-6-yl)piperidin-1-yl]pyrimidine-4-carboxylic acid;
2-[4-(3-chloro-2-methyl-7-oxo-1,7-dihydro-6H-pyrrolo[2,3-c]pyridin-6-yl)piperidin-1-yl]-1,3-thiazole-5-carboxylic acid;
2-[4-(3-chloro-2-methyl-7-oxo-1,7-dihydro-6H-pyrrolo[2,3-c]pyridin-6-yl)piperidin-1-yl]-4-(methoxymethyl)-1,3-thiazole-5-carboxylic acid;
2-[4-(3-chloro-2-methyl-7-oxo-1,7-dihydro-6H-pyrrolo[2,3-c]pyridin-6-yl)piperidin-1-yl]-1,3-thiazole-4-carboxylic acid;
2-[(3S,4R)-4-(3-chloro-2-methyl-7-oxo-1,7-dihydro-6H-pyrrolo[2,3-c]pyridin-6-yl)-3-fluoropiperidin-1-yl]-1,3-thiazole-5-carboxylic acid;
2-[3,4-cis-4-(3-chloro-2-methyl-7-oxo-1,7-dihydro-6H-pyrrolo[2,3-c]pyridin-6-yl)-3-methoxypiperidin-1-yl]-1,3-thiazole-5-carboxylic acid;
2-[4-(3-chloro-2-methyl-7-oxo-1,7-dihydro-6H-pyrrolo[2,3-c]pyridin-6-yl)piperidin-1-yl]-1,3-benzothiazole-7-carboxylic acid;
ethyl 2-[4-(3-chloro-2-methyl-8-oxo-4,5,6,8-tetrahydropyrrolo[2,3-c]azepin-7(1H)-yl)piperidin-1-yl]-1,3-thiazole-5-carboxylate;
ethyl 2-[4-(3-chloro-2-methyl-8-oxo-4,5,6,8-tetrahydropyrrolo[2,3-c]azepin-7(1H)-yl)piperidin-1-yl]-1,3-thiazole-4-carboxylate;
isopropyl 2-[4-(3-chloro-2-methyl-8-oxo-4,5,6,8-tetrahydropyrrolo[2,3-c]azepin-7(1H)-yl)piperidin-1-yl]isonicotinate;
isopropyl 2-[4-(3-chloro-2-methyl-8-oxo-4,5,6,8-tetrahydropyrrolo[2,3-c]azepin-7(1H)-yl)piperidin-1-yl]-4-methyl-1,3-thiazole-5-carboxylate;
methyl 2-chloro-6-[4-(7-cyano-6-methyl-2,4-dioxo-1,2,4,5-tetrahydro-3H-pyrrolo[3,2-d]pyrimidin-3-yl)piperidin-1-yl]pyrimidine-4-carboxylate;
2-[4-(3-chloro-2-methyl-8-oxo-4,5,6,8-tetrahydropyrrolo[2,3-c]azepin-7(1H)-yl)piperidin-1-yl]-1,3-thiazole-5-carboxylic acid;
2-[4-(3-chloro-2-methyl-8-oxo-4,5,6,8-tetrahydropyrrolo[2,3-c]azepin-7(1H)-yl)piperidin-1-yl]-1,3-thiazole-4-carboxylic acid;
2-[4-(3-chloro-2-methyl-8-oxo-4,5,6,8-tetrahydropyrrolo[2,3-c]azepin-7(1H)-yl)piperidin-1-yl]isonicotinic acid; and
2-[4-(3-chloro-2-methyl-8-oxo-4,5,6,8-tetrahydropyrrolo[2,3-c]azepin-7(1H)-yl)piperidin-1-yl]-4-methyl-1,3-thiazole-5-carboxylic acid;
or a pharmaceutically acceptable salt thereof.

14. A process for preparing a compound of formula (I) as claimed in claim 1, wherein the variables are, unless otherwise stated, as defined in claim 1:
Process a) for compounds of formula (I) wherein X is X¹, X², X³, or X⁴; cyclizing a compound of formula (II): 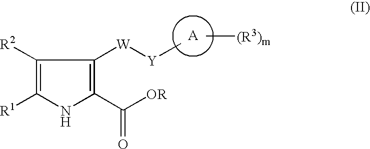
wherein R═C₁₋₄alkyl or hydrogen; W is —NC(O)N, —CH═CHNH—, —N═CH—NH— or —(CH₂)₃—NH— into a compound of formula (I); or
Process b) for compounds of formula (I) wherein X is X²; converting a compound of formula (III): 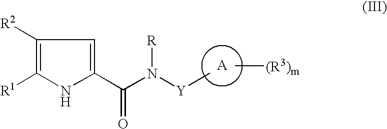
wherein R═—CH₂C(OCH₂CH₂O) into a compound of formula (I); or
Process c) for compounds of formula (I) wherein Y is phenyl; reacting a compound of formula (IV): 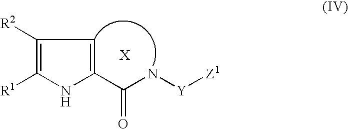
with a compound of formula (V): 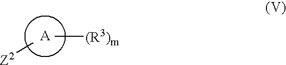
wherein one of Z¹ and Z² is a displaceable group “L” and the other is an organometallic reagent “M”; or
Process d) for compounds of formula (I) wherein Y [[=]] is azetidinyl, piperidinyl or pyrrolidinyl linked to Ring A via the nitrogen in the ring; reacting a compound of formula (VI): 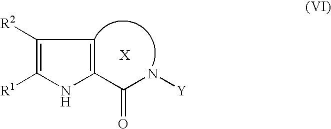
with a compound of formula (VII): 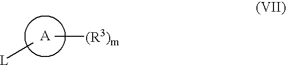
wherein L is a displaceable group;
and thereafter if necessary:
i) converting a compound of the formula (I) into another compound of the formula (I);
ii) removing any protecting groups;
iii) forming a pharmaceutically acceptable salt.

15. A pharmaceutical composition which comprises a compound of the formula (I) or a pharmaceutically-acceptable salt thereof, as claimed in claim 1, and a pharmaceutically-acceptable diluent or carrier.

16-24. (canceled)

25. A method for producing an antibacterial effect in a warm blooded animal in need of such treatment, which comprises administering to said animal an effective amount of a compound of formula (I), or a pharmaceutically-acceptable salt thereof, as claimed in claim 1.

26. A method for inhibition of bacterial DNA gyrase and/or topoisomerase IV in a warm-blooded animal in need of such treatment which comprises administering to said animal an effective amount of a compound of formula (I) or a pharmaceutically acceptable salt thereof, as claimed in claim 1.

27. A method of treating a bacterial infection in a warm-blooded animal in need of such treatment which comprises administering to said animal an effective amount of a compound of formula (I) or a pharmaceutically acceptable salt thereof, as claimed in claim 1.

28. The method as claimed in claim 27 wherein the bacterial infection is an infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria.

29. A compound of formula (I), or a pharmaceutically acceptable salt thereof, as claimed in claim 5, wherein Y is selected from phenyl and piperidinyl; wherein the N of said piperidinyl ring is directly attached to Ring A; and further wherein Y may be optionally substituted on carbon by one halo or C₁₋₄alkoxy.

30. A compound of formula (I), or a pharmaceutically acceptable salt thereof, as claimed in claim 29, wherein Ring A is phenyl, thiazolyl, benzothiazolyl, pyrimidinyl or pyridinyl.

31. A compound of formula (I), or a pharmaceutically acceptable salt thereof, as claimed in claim 30, wherein R³ represents a substituent on carbon and is selected from halo, carboxy, C₁₋₄alkyl, C₁₋₄alkoxy or C₁₋₄alkoxycarbonyl; wherein R³ may be optionally substituted on carbon by one or more R⁶; wherein R⁶ is selected from C₁₋₄alkoxy.

32. A compound of formula (I), or a pharmaceutically acceptable salt thereof, as claimed in claim 31, wherein m is 1 or 2; wherein the values of R³ may be the same or different.

33. A compound of formula (I), or a pharmaceutically acceptable salt thereof, as claimed in claim 7, wherein Y is selected from phenyl and piperidinyl; wherein the N of said piperidinyl ring is directly attached to Ring A; and further wherein Y may be optionally substituted on carbon by one halo or C₁₋₄alkoxy.

34. A compound of formula (I), or a pharmaceutically acceptable salt thereof, as claimed in claim 33, wherein Ring A is phenyl, thiazolyl, benzothiazolyl, pyrimidinyl or pyridinyl.

35. A compound of formula (I), or a pharmaceutically acceptable salt thereof, as claimed in claim 34, wherein R³ represents a substituent on carbon and is selected from halo, carboxy, C₁₋₄alkyl, C₁₋₄alkoxy or C₁₋₄alkoxycarbonyl; wherein R³ may be optionally substituted on carbon by one or more R⁶; wherein R⁶ is selected from C₁₋₄alkoxy.

36. A compound of formula (I), or a pharmaceutically acceptable salt thereof, as claimed in claim 35, wherein m is 1 or 2; wherein the values of R³ may be the same or different.
